(19) United States
(12) Patent Application Publication
(10) Pub. No.: US 2020/0002727 A1
(43) Pub. Date: Jan. 2, 2020

(54) MULTI-SITE SPECIFIC INTEGRATION CELLS FOR DIFFICULT TO EXPRESS PROTEINS

(71) Applicants: LONZA LTD., Visp (CH); PFIZER INC., New York, NY (US)

(72) Inventors: Marc FEARY, Suffolk (GB); Robert J. YOUNG, London (GB); Mark MOFFAT, New York, NY (US); Gerald Fries CASPERSON, New York, NY (US); Heather Laurence JONES, New York, NY (US); Lin ZHANG, New York, NY (US)

(21) Appl. No.: 16/486,363

(22) PCT Filed: Feb. 17, 2018
(86) PCT No.: PCT/IB2018/000232

§ 371 Date: Aug. 15, 2019

Related U.S. Application Data
(60) Provisional application No. 62/460,420, filed on Feb. 17, 2017.

(51) Int. Cl. C12N 15/90; C12N 9/12; C12N 15/86; C07K 16/28; C07K 16/30; C07K 14/55; C07K 16/24

(52) U.S. Cl. CPC C12N 15/907; C12N 9/1241; C12Y 207/07; C12N 15/86; C07K 16/2887; C07K 16/3007; C07K 2319/30; C07K 16/241; C12N 2800/30; C07K 2317/14; C07K 2317/31; C07K 2317/35; C07K 14/55

(57) ABSTRACT

The present disclosure relates to a site-specific integration (SSI) mammalian cell that comprises at least two distinct recombination target sites (RTS) wherein two RTS are chromosomally-integrated within the NL1 locus or the NL2 locus. The disclosure also relates to a SSI mammalian cell comprising at least four distinct RTS wherein two RTS are chromosomally-integrated within the NL1 or the NL2 locus and two RTS are chromosomally-integrated within a separate locus. The disclosure also relates to methods for using the SSI mammalian cell to produce recombinant protein expression cell lines that can express difficult to express proteins.

### SEQUENCE LISTING

The instant application contains a Sequence Listing which has been submitted electronically in ASCII format and is hereby incorporated by reference in its entirety. Said ASCII copy, created on Mar. 28, 2018, is named 0132-0028WO1_SL.txt and is 8,508,333 bytes in size.

### FIELD OF THE INVENTION

The present disclosure relates to a site-specific integration (SSI) mammalian cell that comprises at least two distinct recombination target sites (RTS) wherein two RTS are chromosomally-integrated within the NL1 locus or the NL2 locus. The disclosure also relates to a SSI mammalian cell comprising at least four distinct RTS wherein two RTS are chromosomally-integrated within the NL1 or the NL2 locus and two RTS are chromosomally-integrated within a separate locus. The disclosure also relates to methods for using the SSI mammalian host cell line to produce recombinant protein expression cell lines that can additionally express difficult to express proteins.

### BACKGROUND OF THE INVENTION

Biopharmaceuticals, both novel and biosimilar, continue to see high demand and increasing sales revenues. The increased prevalence of novel format, non-monoclonal antibody (mAb), recombinant proteins (rP), many of which may be classed as difficult to express (DtE) may pose challenges to the delivery of therapeutics in the biopharmaceutical industry. DtE proteins can be associated with lower than expected titres or problems with expression of multiple chains, ancillary proteins, product recovery or purification (Pybus et al. Biotechnol. Bioeng. 111:372-85 (2014)). Examples of DtE proteins include but are not limited to Fc-fusion proteins, bi- and tri-specific MAbs, enzymes, membrane receptors, and bi-specific T-cell engager BITE® (Micromet AG, Munich, Germany) molecules and selected mAbs.

One reported method for integrating recombinant protein (rP) expression cassettes into a host cell genome relies on random integration (RI)—a process by which DNA is introduced into a cell and incorporated at existing double strand breaks (DSB) found in the genome. Consequently, when using such a method, both the number of gene copies integrated and the expression characteristics at integration sites (position variegation effect) can be highly variable. This could be problematic for DtE proteins for a number of reasons. For example, for some multi-chain proteins it could be difficult to control the number of integrated copies of genes encoding individual chains or ancillary genes, vector size limitations could impede the expression of multi-chain proteins, transfecting multiple vectors could result in low integration efficiency and high expression of some DtEs could result in toxicity.

Another method for integrating rP expression cassettes is by using site-specific integration (SSI). “Landing pads” located in the genomes of SSI cell lines can utilize recombination target sites (RTS) derived from site-specific recombinase systems such as the Saccharomyces cerevisiae-derived FLP-Frt system or the bacteriophage P1 derived Cre-loxP system. In these systems, the recombination enzyme or recombinase is responsible for recombination events between donor and target DNA containing compatible recombination sites (Frt or loxP respectively) (see, e.g., Wirth et al. Curr. Op. in Biotech. 18:411-9 (2007)). By using distinct recombination sites at the 5′ and 3′ ends of the cassette to be exchanged (in both donor and target DNA) it is possible to ensure the recombination occurs in a directional manner and that only the preferred cassette region is exchanged. Thus, SSI may be advantageous over random integration approaches. The process of integrating cassettes in SSI cell lines is referred to as recombinase-mediated cassette-exchange (RMCE). Practically, cell line construction for recombinant protein production using RMCE generally involves co-transfection of an expression vector encoding the recombinase along with the targeting expression vector, containing the gene of interest (encoding the rP) and a selection marker flanked by recombinase targeting sequences.

SSI-generated cell lines that use a single landing pad can also have limitations. For example, such an approach usually, by design, results in a low number of integrated gene copies that could indirectly limit rP production recombinant protein expression titres. If production of multiple proteins in an SSI host cell line containing a single landing pad is required for rP production, all of the required genes might need to be included into a single vector. One method to increase integrated copies of recombinant genes is accumulative SSI (sometimes called stacking or multiplexing, see, e.g., Kameyama et al. Biotechnol. Bioeng. 105:1106-14 (2010), Kawabe et al. Cytotechnology 64:267-79 (2012) and Turan et al. J. Mol. Biol. 402:52-69 (2010)). With such a method, repeated rounds of RMCE can be used to load up a single site sequentially with multiple copies of rP expression cassettes. Landing pads in this type of SSI host cell line are not independently addressable and require repeated rounds of RMCE which takes additional time for each cycle.

Various publications are cited herein, the disclosures of which are incorporated by reference herein in their entireties.

### BRIEF DESCRIPTION OF THE INVENTION

A need exists to generate SSI host cell lines that overcome the limitation of SSI hosts with only a single genome insertion site. The present invention fulfills this need by using tandem SSI landing pads—where two or more landing pads are integrated at the same loci may overcome the limitations of cumulative site-specific integration. In such a system, the landing pads are independently addressable (due to recombination site and selection marker choice).

In some embodiments, the present disclosure provides a mammalian cell comprising at least two distinct RTS wherein two RTS are chromosomally-integrated within the NL1 locus or the NL2 locus. In some embodiments, the cell comprises two distinct RTS. In some embodiments, the two distinct RTS are chromosomally-integrated within the NL1 locus. In some embodiments, the two distinct RTS are chromosomally-integrated within the NL2 locus. In some embodiments, the cell comprises four distinct RTS. In some embodiments, the four distinct RTS are chromosomally-integrated on the same locus. In some embodiments, two distinct RTS are chromosomally-integrated on a separate locus. In some embodiments, the separate locus is the Fer1L4 locus. In some embodiments, two distinct RTS are chromosomally-integrated within the NL1 locus, and two distinct RTS are chromosomally-integrated within the NL2 locus. In some embodiments, the cell comprises six distinct RTS. In some embodiments, at least four distinct RTS are chromosomally-integrated on the same locus. In some embodiments, at least two distinct RTS are chromosomally-integrated within the NL1 locus or the NL2 locus, and at least two distinct RTS are chromosomally-integrated on a separate locus. In some embodiments, the separate locus is the Fer1L4 locus. In some embodiments, at least two distinct RTS are chromosomally-integrated within the NL1 locus, and at least two distinct RTS are chromosomally-integrated within the NL2 locus. In some embodiments, at least one of the RTS is an Frt site, a lox site, a rox site, or an att site. In some embodiments, at least one of the RTS is selected from among SEQ ID Nos.: 1-30. In some embodiments, the cell is a mouse cell, a human cell, a Chinese hamster ovary (CHO) cell, a CHO-K1 cell, a CHO-DXB11 cell, a CHO-DG44 cell, a CHOK1SV™ cell including all variants, a CHOK1SV GS-KO™ (glutamine synthetase knockout) cell including all variants, a HEK cell, a HEK293 cell including adherent and suspension-adapted variants, a HeLa cell, or a HT1080 cell. In some embodiments, the cell is a HEK cell.

In some embodiments, the cell comprises a first gene of interest, wherein the first gene of interest is chromosomally-integrated. In some embodiments, the first gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the DtE protein is selected from the group consisting of Fc-fusion protein, an enzyme, a membrane receptor, a bi-specific T-cell engager (BITE® Micromet AG, Munich, Germany), or a monoclonal antibody. In some embodiments, the monoclonal antibody is a bi-specific monoclonal antibody or a tri-specific monoclonal antibody. In some embodiments, the first gene of interest is located between two of the RTS. In some embodiments, the first gene of interest is located within the NL1 locus. In some embodiments, the cell comprises a second gene of interest, wherein the second gene of interest is chromosomally-integrated. In some embodiments, the second gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the DtE protein is selected from the group consisting of a Fc-fusion protein, an enzyme, a membrane receptor, a bi-specific T-cell engager (BITE®), or a monoclonal antibody. In some embodiments, the second gene of interest is located between two of the RTS. In some embodiments, the second gene of interest is located within the NL1 locus or the NL2 locus. In some embodiments, the first gene of interest is located within the NL1 locus, and the second gene of interest is located within the NL2 locus. In some embodiments, the cell comprises a third gene of interest, wherein the third gene of interest is chromosomally-integrated. In some embodiments, the third gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the third gene of interest is located between two of the RTS. In some embodiments, the third gene of interest is located within the NL1 locus or the NL2 locus. In some embodiments, the third gene of interest is located within a locus distinct from the NL1 locus and the NL2 locus. In some embodiments, the first gene of interest, the second gene of interest, and the third gene of interest are within three separate loci. In some embodiments, at least one of the first genes of interest, the second gene of interest, and the third gene of interest is within the NL1 locus, and at least one of the first gene of interest, the second gene of interest, and the third gene of interest is within the NL2 locus. In some embodiments, the cell comprises a site-specific recombinase gene. In some embodiments, the site-specific recombinase gene is chromosomally-integrated.

In some embodiments, the present disclosure provides a mammalian cell comprising at least four distinct RTS, wherein the cell comprises (a) at least two distinct RTS are chromosomally-integrated within the NL1 locus or NL2 locus; (b) a first gene of interest is integrated between the at least two RTS of (a), wherein the first gene of interest comprises a reporter gene, a gene encoding a DtE protein, an ancillary gene or a combination thereof; (c) and a second gene of interest is integrated within a second chromosomal locus distinct from the locus of (a), wherein the second gene of interest comprises a reporter gene, a gene encoding a DtE protein, an ancillary gene or a combination thereof.

In some embodiments, the present disclosure provides a mammalian cell comprising at least four distinct RTS, wherein the cell comprises (a) at least two distinct RTS are chromosomally-integrated within the Fer1L4 locus; (b) at least two distinct RTS are chromosomally-integrated within the NL1 locus or the NL2 locus; (c) a first gene of interest is chromosomally-integrated within the Fer1L4 locus, wherein the first gene of interest comprises a reporter gene, a gene encoding a DtE protein, an ancillary gene or a combination thereof; and (d) a second gene of interest is chromosomally-integrated within the within the NL1 locus or NL2 locus of (b), wherein the second gene of interest comprises a reporter gene, a gene encoding a DtE protein, an ancillary gene or a combination thereof.

In some embodiments, the present disclosure provides a mammalian cell comprising at least six distinct RTS, wherein the cell comprises (a) at least two distinct RTS and a first gene of interest are chromosomally-integrated within the Fer1L4 locus; (b) at least two distinct RTS and a second gene of interest are chromosomally-integrated within the NL1 locus; and (c) at least two distinct RTS and a third gene of interest are chromosomally-integrated within the NL2 locus.

In some embodiments, the present disclosure provides a method for producing a recombinant protein producer cell comprising (a) providing a cell that comprises at least at least four distinct RTS and a gene encoding a site-specific recombinase, wherein at least two distinct RTS are chromosomally-integrated within the NL1 locus and at least two distinct RTS are chromosomally-integrated within the NL2 locus; (b) transfecting the cell of (a) with a first vector comprising an exchangeable cassette encoding a first gene of interest and a second vector comprising an exchangeable cassette encoding a second gene of interest; (c) integrating the first exchangeable cassette within the NL1 locus and the second exchangeable cassette within the NL2 locus; and (d) selecting a recombinant protein producer cell comprising the first exchangeable cassette and the second exchangeable cassette integrated into the chromosome. In some embodiments, the first gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the DtE protein consists of a Fc-fusion protein, an enzyme, a membrane receptor, a bi-specific T-cell engager (BITE®), or a monoclonal antibody. In some embodiments, the monoclonal antibody is a bi-specific monoclonal antibody or a tri-specific monoclonal antibody. In some embodiments, the first gene of interest is located between two of the RTS. In some embodiments, the second gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the second gene of interest is located between two of the RTS.

In some embodiments, the present disclosure provides a method for producing a recombinant protein producer cell comprising (a) providing a cell that comprises at least four distinct RTS and a gene encoding a site-specific recombinase, wherein at least two distinct RTS are chromosomally-integrated within the Fer1L4 locus, and at least two distinct RTS are chromosomally-integrated within the NL1 locus or the NL2 locus; (b) transfecting the cell of (a) with a first vector comprising an exchangeable cassette encoding a first gene of interest and a second vector comprising an exchangeable cassette encoding a second gene of interest; (c) integrating the first exchangeable cassette within the Fer1L4 locus and the second exchangeable cassette within the NL1 locus or the NL2 locus; and (d) selecting a recombinant protein producer cell comprising the first exchangeable cassette and the second exchangeable cassette integrated into the chromosome. In some embodiments, the first gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the DtE protein consists of a Fc-fusion protein, an enzyme, a membrane receptor, a bi-specific T-cell engager (BITE®), or a monoclonal antibody. In some embodiments, the monoclonal antibody is a bi-specific monoclonal antibody or a tri-specific monoclonal antibody. In some embodiments, the first gene of interest is located between two of the RTS. In some embodiments, the second gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the second gene of interest is located between two of the RTS.

In some embodiments, the present disclosure provides a method for producing a recombinant protein producer cell comprising (a) providing a cell that comprises at least at least six distinct RTS and a gene encoding a site-specific recombinase, wherein at least two distinct RTS are chromosomally-integrated within the Fer1L4 locus, and at least two distinct RTS are chromosomally-integrated within the NL1 locus, and at least two distinct RTS are chromosomally-integrated within the NL2 locus; (b) transfecting the cell of (a) with a first vector comprising an exchangeable cassette encoding a first gene of interest, a second vector comprising an exchangeable cassette encoding a second gene of interest, and a third vector comprising an exchangeable cassette encoding a third gene of interest; (c) integrating the first exchangeable cassette within the Fer1L4 locus, the second exchangeable cassette within the NL1 locus, and the third exchangeable cassette within the NL2 locus; and (d) selecting a recombinant protein producer cell comprising the first exchangeable cassette, the second exchangeable cassette and the third exchangeable cassette integrated into the chromosome.

### BRIEF DESCRIPTION OF THE DRAWINGS

FIG. 1 is a schematic for a method of recombinase-mediated cassette-exchange (RMCE) using a multisite site-specific integration (SSI) host cell line with 2 independently addressable landing pads. The number of landing pads is restricted by the availability of suitable loci, incompatible Frt recombinase sites (e.g. wild-type Frt (F), Frt F5 (F5), Frt F14 (F14) and Frt F15 (F15)) and selection markers (e.g. green fluorescent protein (GFP) or Red Fluorescent Protein (RFP)) compatible with such a multiplexing approach.

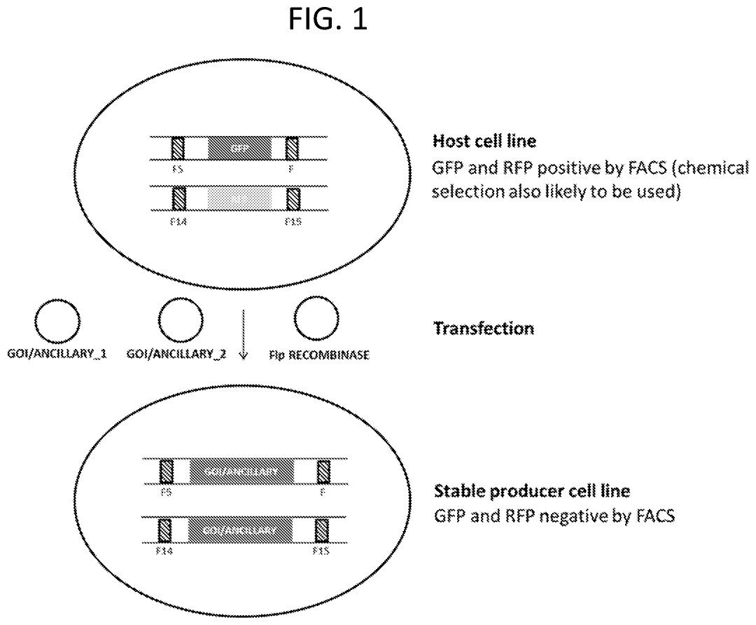

FIG. 2A shows a schematic representation of the landing pad arrangement in a CHOK1SV GS-KO™ (glutamine synthetase knockout) multisite SSI host which contains two different illustrative landing pads (Landing Pad A and Landing Pad B). More details on the chromosomally-integration of these landing pads in single and multisite GS-CHOK1SV™ SSI hosts is given in Table 10. Landing Pad A contains a hygromycin phosphotransferase (Hpt)—enhanced green fluorescent protein (eGFP) fusion gene (Hpt-eGFP). The Hpt-eGFP fusion gene expression is under the control of a SV40 early promoter and has an SV40 polyA sequence. Distinct and different Frt sites are located between the SV40 promoter and Hpt-eGFP gene (F5) and 5′ of the SV40 poly A sequence (F) for RMCE. Landing Pad B contains a puromycin N-acetyltransferase-DsRed (PAC-DsRed) fusion gene under the control of a SV40 promoter and includes a SV40 polyA sequence. A different pair of Frt sites, distinct from those in Landing Pad A, are located between the SV40 early promoter and PAC-DsRed gene (F14) and 5′ of the SV40 polyA sequence (F15). The positioning of the Frt site between the SV40E promoter and selection marker enables it to be used in subsequent rounds of RMCE. Targeting vectors designed for RMCE in SSI single and multisite hosts contain a positive selection marker (e.g. GS) arranged immediately to the 3′ of an Frt site compatible with the destination landing pad (FIG. 2B). The remainder of the vector contains transcription units for the GOI (e.g. mAb) followed by an Frt site compatible to the second Frt site in the landing pad. Targeting vector DNA (FIG. 3A and FIG. 5) is co-transfected with a vector expressing FlpE recombinase (Takata et al., Genes to Cells 16: 7 (2011) (FIG. 3B). Transfected cells are incubated for 24 hours and selection pressure is then applied (e.g. removal of glutamine from culture medium). Successful RMCE is marked by the loss of the Hpt-eGFP gene and replacement with the positive selection marker gene (FIG. 2C). Cells appear dark under the fluorescent microscope or with flow cytometer analysis. Non-exchanged cells which recover in positive selection are easily removed by fluorescence-aided cell sorting.

FIG. 3 shows the pMF25 targeting vector (FIG. 3A) and pMF4 recombinase expression vector (FIG. 3B), used for creating DsRed-producing cell lines in the CHOK1SV GS-KO™ SSI hosts. pMF25 (FIG. 3A) contains a transcription unit incorporating the DsRed gene, flanked by mutant (F5) and wild-type (F) Frt sites. Transcription of DsRed-monomer is driven by the promoter of the hCMV major intermediate early gene 1 (hCMV) and its first intron (Intron A (hCMV Intron)) and the flanking exons encoding the 5′ UTR are denoted as Ex1 and Ex2. Glutamine synthetase cDNA (GS) and SV40 Intron A (SV40 Inton) are arranged immediately to the 3′ of Frt F5 and successful RMCE transcription in driven by SV40E promoter located in the landing pad. pMF4 (FIG. 3B) contains a transcription unit incorporating the FlpE gene (Takata et al., Genes to Cells 16: 7 (2011) driven by the promoter of the hCMV major intermediate early gene 1 (hCMV) and its first intron (Intron A (hCMV Intron)) and the flanking exons encoding the 5′ UTR are denoted as Ex1 and Ex2. In both vectors polyadenylation sequence and β-lactamase are indicated as pA and bla, respectively.

FIG. 4 shows the data from flow cytometry analysis of CHOK1SV GS-KO™ SSI host clones (7878, 8086, 8096, 9113, 9116 and 9115) prior to and following co-transfection with pMF25 and FlpE recombinase encoding vector. Green and yellow fluorescence was measured for the 7878 and 8086 (single site, Fer1L4 Landing Pad A), 8086 and 9113 (single site, NL1 Landing Pad A) and 9116 and 9115 (single site, NL2 Landing Pad A) pre- and post-RMCE (11 days of selection in glutamine-free medium) using a Millipore Guava flow cytometer. eGFP fluorescence was detected in the green channel and DsRed was detected in the yellow channel.

FIG. 5 shows the targeting vector for creating mAb producing cell lines in the CHOK1SV GS-KO™ derived SSI hosts. Vectors pMF26 (A), pMF27 (B) and pMF28 (C) contain transcription units incorporating rituximab, cB72.3 and H31K5 antibody genes (respectively), flanked by mutant (F5) and wild-type (F) Frt sites. Transcription of heavy (HC) and light chain (LC) genes are driven by the promoter of the hCMV major intermediate early gene 1 (hCMV) and its first intron, Intron A (hCMV Intron) and the flanking exons encoding the 5′ UTR are denoted as Ex1 and Ex2. Glutamine synthetase cDNA (GS) and SV40 Intron-polyA (indicated as pA in the FIG. 5) sequences are arranged immediately to the 3′ of Frt F5 to select for on-target integration following RMCE. β-lactamase is indicated as and bla, respectively.

FIG. 6 shows secreted Rituximab, cB72.3 and H31K5 mAb concentrations CHOK1SV GS-KO™ SSI pools grown in batch culture. CHOK1SV GS-KO™ SSI host clone 11434 (single site, Fer1L4 Landing Pad A) was transfected with pMF26 (Rituximab), pMF27 (cB72.3) or pMF28 (H31K5). Replicate (n>3) CHOK1SV GS-KO™ SSI pools were cultured in 8-day batch culture. Secreted rituximab (A), cB72.3 (B) and H31K5 (C) mAb (mg/L) were determined by Protein A HPLC.

FIG. 7 shows the data from flow cytometry analysis of multisite SSI hosts prior to RMCE. Green and yellow fluorescence was measured for the CHOK1SV GS-KO™ host (Host), 11434 (single site, Fer1L4 Landing Pad A), DsRed random integration control and six CHOK1SV GS-KO™ multisite host clones (12151, 12152, 12606, 12607, 12608 and 12609) (multisite sister clones with Landing Pad A in the Fer1L4 loci and Landing Pad B in the NL1 loci) using a Millipore Guava flow cytometer. eGFP fluorescence was detected in the green channel and DsRed was detected in the yellow channel.

FIG. 8 is a schematic of the vectors pCM9 and pCM11 targeted to Fer1L4 and NL1 landing pads in the CHOK1SV GS-KO SSI host. Vectors are as follows: FIG. 8A: pCM9 (E2 crimson expression vector targeting Fer1L4 loci). FIG. 8B: pCM11 (E2 crimson expression vector targeting NL1 loci).

FIG. 9 shows flow cytometry analysis of CHOK1SV GS-KO SSI derived cells targeted with pCM9 and pCM11. Multi CHOK1SV GS-KO SSI clone 12151 (Contains Landing Pad A (FIG. 2A) in Fer1L4 loci and Landing Pad B (FIG. 2A) in NL1 loci) was transfected with either pCM9 (E2 crimson expression vector targeting Fer1L4 loci) and pCM11 (E2 crimson expression vector targeting NL1 loci).

FIG. 10 is a schematic of the Rituximab expression vectors pMF26, pCM38, pCM22 and pAR5 targeted to either Fer1L4 (FIG. 2, Landing Pad A), NL1 (FIG. 2, Landing Pad B) or both. Vectors were as follows: pMF26 (FIG. 10A: Rituximab 1×LC, 1×HC expression vector targeting Fer1L4 loci), pCM38 (FIG. 10B: Rituximab 2×LC, 2×HC expression vector targeting Fer1L4 loci), pCM22 (FIG. 10C: empty expression vector targeting the NL1 loci) and pAR5 (FIG. 10D: Rituximab 1×LC, 1×HC expression vector targeting NL1 loci).

FIG. 11 shows cell specific Rituximab production rates (qmAb) of RMCE pools targeted with pMF26, pCM38, pCM22 and pAR5 following an 8-day batch culture.

FIG. 12 is a schematic of the Cergutuzumab amunaleukin (CEA-IL2v) expression vectors pAB2, pAB5, pCM46 and pAR2, targeted to either Fer1L4 (FIG. 2, Landing Pad A), NL1 (FIG. 2, Landing Pad B) or both. Vectors were as follows: pAB2 (CEA-IL2v LC, HC and HC-IL2 expression vector targeting Fer1L4 loci), pAB5 (CEA-IL2v LC and HC-IL2 expression vector targeting Fer1L4 loci), pCM46 (empty expression vector targeting NL1 loci) and pAR2 (CEA-IL2v HC expression vector targeting NL1 loci).

FIG. 13 is a schematic of SDS-page analysis of RMCE pools expressing Cergutuzumab amunaleukin (CEA-IL2v) following transfection with pAB2, pAB5, pCM46 and pAR2 (FIG. 12). A: Image of a 10% Bis-Tris protein gel generated under non-reduced conditions with lanes 1-9 labelled. Lane 1: Seeblue Plus2 Pre-stained protein standard. Lane 2: Analysis of mock (empty) pool generated by transfecting an empty version of pAB2. Lanes 2-5: Control pools generated with vector expressing only two of three the chain required to generate CEA-IL2v used to assign folding intermediates. Lane 6: analysis of a pools with CEA-IL2v LC, HC and HC-IL2 (pAB2) in Fer1L4 locus. Lane 7: analysis of a pools with CEA-IL2v LC and HC-IL2 in Fer1L4 locus and empty vector (pCM46) in NL1. Lanes 8 and 9: CEA-IL2v LC and HC-IL2 in Fer1L4 locus and CEA-IL2v HC (pAR2) in NL1 locus. B: Histogram summarizing densitometry of bands hypothesized to represent light chain (LC), light chain dimer (2LC), half antibody (1LC, 1HC-IL2) and part assembled Cergutuzumab amunaleukin (2LC, 2HC-IL2 and 2LC, 2HC).

FIG. 14 is a schematic of the Entanercept, Ancillary or destabilized GFP (dsGFP with miR target sequence in 3′UTR) gene vectors targeted to either Fer1L4 (FIG. 2, Landing Pad A), NL1 (FIG. 2, Landing Pad B) or both. Vectors were as follows: pTC1 (FIGS. 14A, B and C: Entanercept expression vector targeting Fer1L4 loci), pCM22 (FIG. 14A: Empty vector targeting NL1), pCM39 (FIG. 14B: Mus musculus SCD1 (mSCD1) expression vector targeting NL1), pCM40 (FIG. 14B: Mus musculus SCD1 codon optimized for Cricetulus griseus (ccmSCD1) expression vector targeting NL1), pCM41 (FIG. 14B: Homo sapiens SCD1 (hSCD1), pCM42 (FIG. 14B: Cricetulus griseus SREBF1 (ccSREBF1) expression vector targeting NL1), pCM43 (FIG. 14C: dsGFP_6n CPEB2A expression vector targeting NL1), pCM44 (FIG. 14C: dsGFP_6n CPEB2B expression vector targeting NL1) and pCM45 (FIG. 14C: dsGFP_6n SRPα expression vector targeting NL1). See Table 11 and Table 12 for details of ancillary genes and sponge sequences.

FIG. 15 Summarizes growth and productivity measurements from 8-day culture of RMCE pools expressing Entanercept with or without ancillary gene expression (See FIG. 14). Data are mean (n=2)±Standard deviation. A: Cell specific production rate (qP). B: Integral of viable cell concentration (IVCC). C: Secreted Entanercept concentration.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited, elements or method steps. It is contemplated that any embodiment discussed in this specification can be implemented with respect to any method, system, host cells, expression vectors, and/or composition of the invention. Furthermore, compositions, systems, cells, and/or vectors of the invention can be used to achieve any of the methods as described herein.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

In some embodiments, the present disclosure provides a mammalian cell comprising at least two distinct RTS wherein the RTS are chromosomally-integrated within the NL1 locus or the NL2 locus.

As referred to herein, the term “mammalian cell” includes cells from any member of the order Mammalia, such as, for example, human cells, mouse cells, rat cells, monkey cells, hamster cells, and the like. In some embodiments, the mammalian cell is a mouse cell, a human cell, a Chinese hamster ovary (CHO) cell, a CHO-K1 cell, a CHO-DXB11 cell, a CHO-DG44 cell, a CHOK1SV™ cell including all variants (e.g. CHOK1SV™ POTELLIGENT®, Lonza, Slough, UK), a CHOK1SV GS-KO™ (glutamine synthetase knockout) cell including all variants, a HEK293 cell including adherent and suspension-adapted variants, a HeLa cell, or a HT1080 cell.

A “nucleic acid,” “nucleic acid molecule,” or “oligonucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. RNA includes, but is not limited to, mRNA, tRNA, rRNA, snRNA, microRNA, miRNA, or MIRNA.

An “amino acid” as used herein refers to a compound containing both a carboxyl (—COOH) and amino (—NH₂) group. “Amino acid” refers to both natural and unnatural, i.e., synthetic, amino acids. Natural amino acids, with their three-letter and single letter abbreviations, include alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “chain” and polypeptide “chain” are used interchangeably herein and refer to a polymeric form of amino acids of a single peptide backbone.

The term “recombinant” when used in reference to a nucleic acid molecule, peptide, polypeptide, or protein means of, or resulting from, a new combination of genetic material that is not known to exist in nature. A recombinant molecule can be produced by any of the well-known techniques available in the field of recombinant technology, including, but not limited to, polymerase chain reaction (PCR), gene cutting (e.g., using restriction endonucleases), and solid state synthesis of nucleic acid molecules, peptides, or proteins. In some embodiments, “recombinant” refers to a viral vector or virus that is not known to exist in nature, e.g. a viral vector or virus that has one or more mutations, nucleic acid insertions, or heterologous genes in the viral vector or virus. In some embodiments, “recombinant” refers to a cell or host cell that is not known to exist in nature, e.g. a cell or host cell that has one or more mutations, nucleic acid insertions, or heterologous genes in the cell or host cell.

An “isolated” polypeptide, protein, peptide, or nucleic acid is a molecule that has been removed from its natural environment. It is also to be understood that “isolated” polypeptides, proteins, peptides, or nucleic acids may be formulated with excipients such as diluents or adjuvants and still be considered isolated.

The terms “sequence identity” or “% identity” in the context of nucleic acid sequences or amino acid sequences refers to the percentage of residues in the compared sequences that are the same when the sequences are aligned over a specified comparison window. A comparison window can be a segment of at least 10 to over 1000 residues in which the sequences can be aligned and compared. Methods of alignment for determination of sequence identity are well-known in the art can be performed using publicly available databases such as BLAST (blast.ncbi.nlm.nih.gov/Blast.cgi.).

In some embodiments, polypeptides or nucleic acid molecules have at least about 70%, at least about 75%, at least about 80%, at least about 85%, at least about 90%, at least about 95%, at least about 97%, at least about 98%, at least about 99% or about 100% sequence identity with a reference polypeptide or nucleic acid molecule, respectively (or a fragment of the reference polypeptide or nucleic acid molecule). In certain embodiments of the disclosure, polypeptides or nucleic acid molecules have at least 70%, at least 75%, at least 80%, at least 85%, at least 90%, at least 95%, at least 97%, at least 98%, or at least 99% or 100% sequence identity with a reference polypeptide or nucleic acid molecule, respectively (or a fragment of the reference polypeptide or nucleic acid molecule). In some embodiments, polypeptides or nucleic acid molecules have about 90%, 91%, 92%, 93%, 94%, 95%, 96%, 97%, 98%, 99%, 99.5% sequence identity with a reference polypeptide or nucleic acid molecule, respectively.

A “gene” refers to an assembly of nucleotides that encode a polypeptide, and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated in the host cell genome with multiple copies. In some embodiments, genes are integrated in the host cell genome at predefined copy numbers.

As referred to herein, the term “regulatory element” refers to a genetic element which controls some aspect of the expression of nucleic acid sequences. As used herein, “promoter,” “promoter sequence,” or “promoter region” refers to a DNA regulatory region/sequence capable of binding RNA polymerase and involved in initiating transcription of a downstream coding or non-coding sequence. In some examples of the present disclosure, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the gene expression, e.g., in the host cell or vectors of the present disclosure. In some embodiments, the promoter is not a leaky promoter, i.e., the promoter is not constitutively expressing any one of the gene products as described herein.

As used herein, the term “heterologous promoter” refers to such a regulatory element which is derived from a different species than the gene to which it is operably linked. In some embodiments, the heterologous promoter is derived from a prokaryotic system. In some embodiments, the heterologous promoter is derived from a eukaryotic system. In some embodiments, the disclosure provides for a cell in which one or more heterologous promoters are chromosomally-integrated into the host cell genome.

As referred to herein, the terms “in operable combination,” “in operable order,” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. In some embodiments, a gene of interest is operably linked to a promoter, wherein the gene of interest is chromosomally-integrated into the host cell. In some embodiments, the gene of interest is operably linked to a heterologous promoter; where in the gene of interest is chromosomally-integrated into the host cell. In some embodiments, an ancillary gene is operably linked to a promoter, wherein the ancillary gene is chromosomally-integrated into the host cell genome. In some embodiments, the ancillary gene is operably linked to a heterologous promoter; where in the ancillary gene is chromosomally-integrated into the host cell genome. In some embodiments, a gene encoding a DtE protein is operably linked to a promoter, wherein the gene encoding a DtE protein is chromosomally-integrated into the host cell genome. In some embodiments, the gene encoding a DtE protein is operably linked to a heterologous promoter, where in the gene encoding a DtE protein is chromosomally-integrated into the host cell genome. In some embodiments, a recombinase gene is operably linked to a promoter, wherein the recombinase gene is chromosomally-integrated into the host cell. In some embodiments, the recombinase gene is operably linked to a promoter, where in the recombinase gene is not integrated into the host cell genome. In some embodiments, a recombinase gene is operably linked to a heterologous promoter, wherein the recombinase gene is not chromosomally-integrated into the host cell genome. In some embodiments, the recombinase gene is operably linked to a heterologous promoter, wherein the recombinase gene is not chromosomally-integrated into the host cell genome.

In some embodiments, regulatory elements operably link gene expression to the presence of an exogenously supplied ligand. In some embodiments, a gene of interest is operably linked to a promoter, wherein the promoter operably links gene expression to the presence of an exogenously supplied ligand and wherein the gene of interest is chromosomally-integrated into the host cell genome. In some embodiments, an ancillary gene is operably linked to a promoter, wherein the promoter operably links gene expression to the presence of an exogenously supplied ligand and wherein the ancillary gene is chromosomally-integrated into the host cell. In some embodiments, a gene encoding a DtE protein is operably linked to a promoter, wherein the promoter operably links gene expression to the presence of an exogenously supplied ligand and wherein the gene encoding a DtE protein is chromosomally-integrated into the host cell genome. In some embodiments, a recombinase gene is operably linked to a promoter, wherein the promoter operably links gene expression to the presence of an exogenously supplied ligand and wherein the recombinase gene is chromosomally-integrated into the host cell genome. In some embodiments, a recombinase gene is operably linked to a promoter, wherein the promoter operably links gene expression to the presence of an exogenously supplied ligand and wherein the recombinase gene is not chromosomally-integrated into the host cell genome.

As referred to herein, the term “chromosomally-integrated” or “chromosomal integration” refers to the stable incorporation of a nucleic acid sequence into the chromosome of a host cell, e.g. a mammalian cell. i.e., a nucleic acid sequence that is chromosomally-integrated into the genomic DNA (gDNA) of a host cell, e.g. a mammalian cell. In some embodiments, a nucleic acid sequence that is chromosomally-integrated is stable. In some embodiments, a nucleic acid sequence that is chromosomally-integrated is not located on a plasmid or a vector. In some embodiments, a nucleic acid sequence that is chromosomally-integrated is not excised. In some embodiments, chromosomal integration is mediated by the clustered regularly interspaced short palindromic repeats (CRISPR) and CRISPR associated protein (Cas) gene editing system (CRISPR/CAS).

As referred to herein, the term “chromosomal locus” refers to defined location of nucleic acids on the chromosome of the cell that may comprise at least one gene. In some embodiments, the chromosomal locus comprises about 500 base pairs to about 100,000 base pairs, about 5,000 base pairs to about 75,000 base pairs, about 5,000 base pairs to about 60,000 base pairs, about 20,000 base pairs to about 50,000 base pairs, about 30,000 base pairs to about 50,000 base pairs, or about 45,000 base pairs to about 49,000 base pairs. In some embodiments, the chromosomal locus extends up to about 100 base pairs, about 250 base pairs, about 500 base pairs, about 750 base pairs, or about 1000 base pairs to the 5′ or the 3′ end of the defined nucleic acid sequence. In some embodiments, the chromosomal locus comprises an endogenous nucleic acid sequence. In some embodiments, the chromosomal locus comprises an exogenous nucleotide sequence having been integrated into the chromosome using methods known to one of the art of molecular biology. In some embodiments, the chromosomal locus comprises a nucleotide sequence in the genome of a host cell which provides for a strong and stable production of a heterologous protein encoded by a gene of interest integrated within the chromosomal locus. In some embodiments, the chromosomal locus comprises a nucleotide sequence in the genome of a host cell which provides for a strong and stable viral gene expression. In some embodiments, the chromosomal locus comprises a nucleotide sequence in the genome of a host cell which provides efficient site-specific integration. In some embodiments, the chromosomal locus comprises the NL1 locus, the NL2 locus, the NL3 locus, the NL4 locus, the NL5 locus, or the NL6 locus as described in Table 1. In some embodiments, the disclosure is directed to a mammalian cell comprising at least two distinct RTS wherein two RTS are chromosomally-integrated within the NL1 locus, the NL2 locus, the NL3 locus, the NL4 locus, the NL5 locus, or the NL6 locus as described in Table 1. In some embodiments, the disclosure is directed to a mammalian cell comprising at least two distinct RTS wherein two RTS are chromosomally-integrated within the NL1 locus or the NL2 locus as described in Table 1.

TABLE 1

| | | | | Loci Location in Cricetulus griseus | | | | | |
|---|---|---|---|---|---|---|---|---|---|
| | | | | NCBI Reference | | | Loci Location in Homo sapien | | |
| | Loci Gene Annotation | | | Sequence⁽¹⁾ | | Landing | | | Landing |
| New | | Within | | (Cricetulus griseus) | Vector | Pad | Chromo- | Vector | Pad |
| Loci | 5′gene | gene | 3′ gene | Sequence Number | Location | Location | some | Location) | Location |
| Fer1 | Ergic3 | Fer1L4 | Spag4 | gi\|351517716\| | N/A | 24894- | Chr 20 | 33613757- | 33630003- |
| | | | | ref\|NW_003613833\| | | 26755 | | 33614117 | 33629572 |
| | | | | Scaffold 1492 | | | | | |
| | | | | SEQ ID NO: 31 | | | | | |
| NL1 | Col5a2 | No | Col5a2 | gi\|351515650\| | 143287 | 133598 | Chr 2 | 189725696- | 189725180- |
| | (pseudo) | annotated | | ref\|NW_003615899.1\| | | | | 189726735 | 189727852 |
| | | gene | | Scaffold 2552 | | | | | |
| | | | | SEQ ID NO: 32 | | | | | |
| NL2 | LOC103 | Naa15 | Rab33b | gi\|351517715\| | 1303708 to | 1313999 | Chr 4 | 140474660- | 140483934- |
| | 162114 | (Intron | | ref\|NW_003613834.1\| | 1308873 | | | 140474867 | 140483457 |
| | | between | | Scaffold2422 | | | | | |
| | | Exon | | SEQ ID NO: 33 | | | | | |
| | | 5 & 6) | | | | | | | |
| NL3 | CJO004127.1 | No | Mthfd2 | gi\|351516540\| | 279558 | N/A | Chr 2 | 74277198- | N/A |
| | | annotated | | ref\|NW_003615009.1\| | | | | 74297932 | |
| | | gene | | Scaffold3924 | | | | | |
| | | | | SEQ ID NO: 34 | | | | | |
| NL4 | MORF4L1 | No | No | gi\|351516248\| | 173850 | N/A | Chr 15 | 76952458 | N/A |
| | | annotated | anno- | ref\|NW_003615301.1\| | | | | | |
| | | gene | tated | Scaffold502 | | | | | |
| | | | gene | SEQ ID NO: 35 | | | | | |
| NL5 | ZNHIT1 | No | Tatc1 | gi\|351517397\| | 779010 | N/A | Chr 7 | 100648176- | N/A |
| | | annotated | | ref\|NW_003614152.1\| | | | | 100653983 | |
| | | gene | | Scaffold3667 | | | | | |
| | | | | SEQ ID NO: 36 | | | | | |
| NL6 | Arhgef28 | Utp15 | Ankra | gi\|351516957\| | 225021 | N/A | Chr 5 | 73084488- | N/A |
| | | | | ref\|NW_003614592.1\| | | | | 73084783 | |
| | | | | Scaffold7019 | | | | | |
| | | | | SEQ ID NO: 37 | | | | | |

One of skill in the art will recognize that the term “integrated within the NL1 locus” or “integrated within the NL2 locus” will include integration into any part of the locus, and it not limited to just the indicated genomic coordinates. One of skill in the art will recognize that the term “integrated within the NL1 locus” or “integrated within the NL2 locus” would also include corresponding loci in corresponding organisms. Thus, in some embodiments, the term “integrated within the NL1 locus” or “integrated within the NL2 locus” will include integration within about 50,000 bp, within about 40,000 bp, within about 30,000 bp, within 20,000 bp or within 10,000 bp of the indicated genomic coordinates.

In some embodiments, the disclosure is directed to a mammalian cell comprising at least two distinct RTS wherein two RTS are chromosomally-integrated into a chromosomal locus selected from Fer1L4 (see e.g. U.S. patent application Ser. No. 14/409,283), ROSA26, HGPRT, DHFR, COSMC, LDHA, or MGAT1. In some embodiments, the chromosomal locus comprises the first intron of MID1 on the X chromosome. In some embodiments, the chromosomal locus comprises an enhanced expression and stability region (EESYRs see, e.g. U.S. Pat. No. 7,771,997). In some embodiments, at least a portion of a gene in the native chromosomal locus is deleted.

A “vector” or “expression vector” is a replicon, such as a plasmid, phage, virus, or cosmid, to which another DNA segment may be attached to bring about the replication and/or expression of the attached DNA segment in a cell. “Vector” includes episomal (e.g., plasmids) and non episomal vectors. In some embodiments of the present disclosure the vector is an episomal vector, which is removed/lost from a population of cells after a number of cellular generations, e.g., by asymmetric partitioning. The term “vector” includes both viral and non-viral means for introducing a nucleic acid molecule into a cell in vitro, in vivo, or ex vivo. The term vector may include synthetic vectors. Vectors may be introduced into the desired host cells by well-known methods, including, but not limited to, transfection, transduction, cell fusion, and lipofection. Vectors can comprise various regulatory elements including promoters.

As used herein, the term “exchangeable cassette” or “cassette” is a type of mobile genetic element that contains a gene and a recombination site. In some embodiments, the exchangeable cassette comprises at least two RTS. In some embodiments, the exchangeable cassette comprises a reporter gene or a selection gene. In some embodiments, a cassette is exchanged through recombinase-mediated cassette-exchange (RMCE).

In some embodiments, site-specific integration is used to introduce one or more genes into a host cell chromosome. See, e.g., Bode et al., Biol. Chem. 381:801-813 (2000), Kolb, Cloning and Stem Cells 4:381-392 (2002) and Coates et al., Trends in Biotech. 23:407-419 (2005), each of which is incorporated by reference. In some embodiments, “site-specific integration” refers to integration of a nucleic acid sequence into a chromosome at a specific site. In some embodiments, site-specific integration can also mean “site-specific recombination.” In some embodiments, “site-specific recombination” refers to the rearrangement of two DNA partner molecules by specific enzymes performing recombination at their cognate pairs of sequences or target sites. Site-specific recombination, in contrast to homologous recombination, requires no DNA homology between partner DNA molecules, is RecA-independent, and does not involve DNA replication at any stage. In some embodiments, site-specific recombination uses a site-specific recombinase system to achieve site-specific integration of nucleic acids in host cells, e.g. mammalian cells. A recombinase system typically consists of three elements: two specific DNA sequences (recombination target sites) and a specific enzyme (recombinase). The recombinase catalyzes a recombination reaction between the specific recombination sites.

A recombinase enzyme, or recombinase, is an enzyme that catalyzes recombination in site-specific recombination. In some embodiments of the disclosure, the recombinase used for site-specific recombination is derived from a non-mammalian system. In some embodiments, the recombinase is derived from bacteria, bacteriophage, or yeast.

In some embodiments, a nucleic acid sequence encoding a recombinase is integrated into the host cell, e.g. mammalian cell. In some embodiments, a nucleic acid sequence encoding a recombinase is delivered to the host cell by methods known to molecular biology. In some embodiments, a recombinase polypeptide sequence can be delivered to the cell directly. In some embodiments, the recombinase is a Cre recombinase, a FLP recombinase, a Dre recombinase, a KD recombinase, a B2B3 recombinase, a Hin recombinase, a Tre recombinase, a λ integrase, a HK022 integrase, a HP1 integrase, a γδ resolvase/invertase, a ParA resolvase/invertase, a Tn3 resolvase/invertase, a Gin resolvase/invertase, a φC31 integrase, a BxB1 integrase, a R4 integrase or another functional recombinase enzyme. See, e.g., Thorpe & Smith, Proc. Nat'l. Acad. Sci. USA 95: 5505-5510 (1998); Kuhstoss & Rao, J. Mol. Biol. 222: 897-890 (1991); U.S. Pat. No. 5,190,871; Ow & Ausubel, J. Bacteriol. 155: 704-713 (1983); Matsuura et al., J. Bacteriol. 178: 3374-3376 (1996); Sato et al., J. Bacteriol. 172: 1092-1098 (1990); Stragier et al., Science 243: 507-512 (1989); Carrasco et al., Genes Dev. 8: 74-83 (1994); Bannam et al., Mol. Microbiol. 16: 535-551 (1995); Crelin & Rood, J. Bacteriol. 179: 5148-5156 (1997).

In some embodiments, a recombinase as described herein is a FLP recombinase. A FLP recombinase is a protein which catalyzes a site-specific recombination reaction that is involved in amplifying the copy number of the 2 plasmid of Saccharomyces cerevisiae during DNA replication. In some embodiments, the FLP recombinase of the present disclosure is derived from species of the genus Saccharomyces. In some embodiments, the FLP recombinase is derived from Saccharomyces cerevisiae. In some embodiments, the FPL recombinase is derived from a strain of Saccharomyces cerevisiae. In some embodiments, the FLP recombinase is a thermostable, mutant FLP recombinase. In some embodiments, the FLP recombinase is FLP1 or FLPe. In some embodiments, the nucleic acid sequence encoding the FLP recombinase comprises human optimized codons.

In some embodiments, the recombinase is a Cre recombinase. Cre (causes recombination) is a member of the Int family of recombinases (Argos et al. (1986) EMBO J. 5:433) and has been shown to perform efficient recombination of lox sites (locus of X-ing over) not only in bacteria but also in eukaryotic cells (Sauer (1987)Mol. Cell. Biol. 7:2087; Sauer and Henderson (1988) Proc. Natl Acad. Sci. 85:5166). In some embodiments, the Cre recombinase as described and used herein is derived from bacteriophage. In some embodiments, the Cre recombinase is derived from P1 bacteriophage.

As referred to herein, the terms “site-specific integration site,” “recombination target site,” “RTS,” and “site-specific recombinase target site” refer to a short, e.g. less than about 60 base pairs, nucleic acid site or sequence which is recognized by a site-specific recombinase and which become the crossover regions during the site-specific recombination event. In some embodiments, the recombination target site is less than about 60 base pairs, less than about 55 base pairs, less than about 50 base pairs, less than about 45 base pairs, less than about 40 base pairs, less than about 35 base pairs, or less than about 30 base pairs. In some embodiments, the recombination target site is about 30 to about 60 base pairs, about 30 to about 55 base pairs, about 32 to about 52 base pairs, about 34 to about 44 base pairs, about 32 base pairs, about 34 base pairs, or about 52 base pairs. Examples of site-specific recombinase target sites include, but are not limited to, lox sites, rox sites, frt sites, att sites and dif sites. In some embodiments, recombination target sites are nucleic acids having substantially the same sequence as set forth in SEQ ID NOs.: 1-30.

In some embodiments, the RTS is a lox site selected from Table 2. As referred to herein, the term “lox site” refers to a nucleotide sequence at which a Cre recombinase can catalyze a site-specific recombination. A variety of non-identical lox sites are known to the art. The sequences of the various lox sites are similar in that they all contain identical 13-base pair inverted repeats flanking an 8-base pair asymmetric core region in which the recombination occurs. It is the asymmetric core region that is responsible for the directionality of the site and for the variation among the different lox sites. Illustrative (non-limiting) examples of these include the naturally occurring loxP (the sequence found in the P1 genome), loxB, loxL and loxR (these are found in the E. coli chromosome) as well as several mutant or variant lox sites such as loxP 511, loxΔ86, loxΔ 117, loxC 2, loxP 2, loxP 3 and loxP 23. In some embodiments, a lox recombination target site is a nucleic acid having at least 90%, 91%, 92%, 93%, 94%, 95%, 96%, 97%, 98%, 99%, or 100% sequence identity to the sequences found in Table 2.

TABLE 2

| Name | Identifier | Sequence |
|---|---|---|
| lox P | SEQ ID NO.: 1 | ATAACTTCGTATAATGTATGCTATA |
| | | CGAAGTTAT |
| loxP 511 | SEQ ID NO.: 2 | ATAACTTCGTATAATGTATACTATA |
| | | CGAAGTTAT |
| loxP 2272 | SEQ ID NO.: 3 | ATAACTTCGTATAAAGTATCCTATA |
| | | CGAAGTTAT |
| loxP 5171 | SEQ ID NO.: 4 | ATAACTTCGTATAATGTGTACTATA |
| | | CGAAGTTAT |
| loxP 2272 | SEQ ID NO.: 5 | ATAACTTCGTATAGGATACTTTATA |
| (V) | | CGAAGTTAT |
| pLox2+ | SEQ ID NO.: 6 | ATAACTTCGTATAATGTATGCTATA |
| | | CGAAGTTAT |
| loxC 2 | SEQ ID NO.: 28 | ACAACTTCGTATAATGTATGCTATA |
| | | CGAAGTTAT |
| loxP 3 | SEQ ID NO.: 29 | TACCGTTCGTATAGTATAGTATATA |
| | | CGAAGTTAT |
| loxP 23 | SEQ ID NO.: 30 | TACCGTTCGTATAGTATAGTATATA |
| | | CGAACGGTA |

In some embodiments, the RTS is a lox site selected from loxΔ86, loxΔ117, loxC2, loxP 2, loxP 3 and loxP 23.

In some embodiments, the RTS is a Frt site selected from Table 3. As referred to herein, the term “Frt site” refers to a nucleotide sequence at which the product of the FLP gene of the yeast 2 μm plasmid, FLP recombinase, can catalyze a site-specific recombination. A variety of non-identical Frt sites are known to the art. The sequences of the various Frt sites are similar in that they all contain identical 13-base pair inverted repeats flanking an 8-base pair asymmetric core region in which the recombination occurs. It is the asymmetric core region that is responsible for the directionality of the site and for the variation among the different Frt sites. Illustrative (non-limiting) examples of these include the naturally occurring Frt (F), and several mutant or variant Frt sites such as Frt F1 and Frt F2. In some embodiments, the Frt recombination target site is a nucleic acid having at least 90%, 91%, 92%, 93%, 94%, 95%, 96%, 97%, 98%, 99%, or 100% sequence identity to the sequences found in Table 3.

TABLE 3

| Name | Identifier | Sequence |
|---|---|---|
| F | SEQ ID NO.: 7 | GAAGTTACTATTCCGAAGTTCCTATTCTCTA |
| | | GAAAGTATAGGAACTTC |
| F1 | SEQ ID NO.: 8 | GAAGTTACTATTCCGAAGTTCCTATTCTCTA |
| | | GATAGTATAGGAACTTC |
| F2 | SEQ ID NO.: 9 | GAAGTTACTATTCCGAAGTTCCTATTCTCTA |
| | | CTTAGTATAGGAACTTC |
| F3 | SEQ ID NO.: 10 | GAAGTTACTATTCCGAAGTTCCTATTCTTCA |
| | | AATAGTATAGGAACTTC |
| F4 | SEQ ID NO.: 11 | GAAGTTACTATTCCGAAGTTCCTATTCTCTA |
| | | GAAGGTATAGGAACTTC |
| F5 | SEQ ID NO.: 12 | GAAGTTCCTATTCCGAAGTTCCTATTCTTCA |
| | | AAAGGTATAGGAACTTC |
| F6 | SEQ ID NO.: 13 | GAAGTTCCTATTCCGAAGTTCCTATTCTTCA |
| | | AAAAGTATAGGAACTTC |
| F7 | SEQ ID NO.: 14 | GAAGTTCCTATTCCGAAGTTCCTATTCTTCA |
| | | ATAAGTATAGGAACTTC |
| F14 | SEQ ID NO.: 15 | GAAGTTCCTATTCCGAAGTTCCTATTCTATC |
| | | AGAAGTATAGGAACTTC |
| F15 | SEQ ID NO.: 16 | GAAGTTCCTATTCCGAAGTTCCTATTCTTAT |
| | | AGGAGTATAGGAACTTC |
| Ff61 | SEQ ID NO.: 17 | GAAGTTACTATTCCGAAGTTCCTATACTTTC |
| | | TGGAGAATAGGAACTTC |
| F2151 | SEQ ID NO.: 18 | GAAGTTACTATTCCGAAGTTCCTATACTCTC |
| | | CAGAGAATAGGAACTTC |
| Fw2 | SEQ ID NO.: 19 | GAAGTTACTATTCCGAAGTTCCTATACTATC |
| | | TACAGAATAGGAACTTC |
| F2161 | SEQ ID NO.: 20 | GAAGTTACTATTCCGAAGTTCCTATACTCTC |
| | | TGGAGAATAGGAACTTC |
| F2262 | SEQ ID NO.: 21 | GAAGTTACTATTCCGAAGTTCCTATACTATC |
| | | TTGAGAATAGGAACTTC |

In some embodiments, the RTS is a rox site selected from Table 4. As referred to herein, the term “rox site” refers to a nucleotide sequence at which a Dre recombinase can catalyze a site-specific recombination. A variety of non-identical rox sites are known to the art. Illustrative (non-limiting) examples of these include roxR and roxF. In some embodiments, a rox recombination target site is a nucleic acid having at least 90%, 91%, 92%, 93%, 94%, 95%, 96%, 97%, 98%, 99%, or 100% sequence identity to the sequences found in Table 4.

TABLE 4

| Name | Identifier | Sequence |
|---|---|---|
| roxF | SEQ ID NO.: 22 | TAACTTTAAATAATGCCAATTATTTAAAG |
| | | TTA |
| roxR | SEQ ID NO.: 23 | TAACTTTAAATAATTGGCATTATTTAAAG |
| | | TTA |

In some embodiments, the RTS is an att site selected from Table 5. As referred to herein, the term “att site” refers to a nucleotide sequence at which a λ integrase or φC31 integrase, can catalyze a site-specific recombination. A variety of non-identical aat sites are known to the art. Illustrative (non-limiting) examples of these include attP, attB, proB, trpC, galT, thrA, and rrnB. In some embodiments, an att recombination target site is a nucleic acid having at least 90%, 91%, 92%, 93%, 94%, 95%, 96%, 97%, 98%, 99%, or 100% sequence identity to the sequences found in Table 5.

TABLE 5

| Name | Identifier | Sequence |
|---|---|---|
| attB | SEQ ID NO.: 24_ | CATCAGGGCGGTCAGGCCGTAGATGTGGAA |
| | | AGAACGGCAGCACGGCGAGGACG |
| attP | SEQ ID NO.: 25 | ATGTGGTCCTTTAGATCCACTGACGTGGGT |
| | | CAGTGTCTCTAAAGGACTCGCG |
| attL | SEQ ID NO.: 26 | CATCAGGGCGGTCAGGCCGTAGATGTGGGT |
| | | CAGTGTCTCTAAAGGACTCGCG |
| attR | SEQ ID NO.: 27_ | ATGTGGTCCTTTAGATCCACTGACGTGGAA |
| | | GAACGGCAGCACGGCGAGGACG |

As referred to herein, the term “distinct recombination target sites” refers to non-identical or hetero-specific recombination target sites. For example, several variant Frt sites exist, but recombination can usually occur only between two identical Frt sites. In some embodiments, distinct recombination target sites refer to non-identical recombination target sites from the same recombination system (e.g. LoxP and LoxR). In some embodiments, distinct recombination target sites refer to non-identical recombination target sites from different recombination systems (e.g. LoxP and Frt). In some embodiments, distinct recombination target sites refer to a combination of recombination target sites from the same recombination system and recombination target sites from different recombination systems (e.g. LoxP, LoxR, Frt, and Frt1).

Various combinations of RTS can be used. In some embodiments, the cell comprises two RTS. In some embodiments, the cell comprises four RTS. In some embodiments, the cell comprises six RTS. In some embodiments, at least one RTS is selected from SEQ ID Nos.: 1-30. In some embodiments, the cell comprises six RTS. In some embodiments, at least one RTS is selected from SEQ ID Nos.: 1-6. In some embodiments, the cell comprises six RTS. In some embodiments, at least one RTS is selected from SEQ ID Nos.: 7-21. In some embodiments, the cell comprises six RTS. In some embodiments, at least one RTS is selected from SEQ ID Nos.: 22-23. In some embodiments, at least one RTS is selected from SEQ ID Nos.: 28-30. In some embodiments, the cell comprises six RTS. In some embodiments, at least one RTS is selected from SEQ ID Nos.: 24-27. In some embodiments, the cell comprising at least four distinct RTS includes the following RTS: LoxP 511, Frt F5, Frt, and LoxP. In some embodiments, the cell comprising at least four distinct RTS includes the following RTS: Frt F14 and Frt F15. In some embodiments, the cell comprising at least four distinct RTS includes the following RTS: LoxP 511, Frt F5, Frt, LoxP, Frt F14 and Frt F15. In some embodiments, the cell comprising at least four distinct RTS can include the following RTS: Frt 1, Frt 2, Frt 3, Frt 4. In some embodiments, the cell comprising at least four distinct RTS includes the following RTS: Frt m5, Frt wt, Frt m14, Frt m15, Frt m7 and Frt m6. In some embodiments, the cell comprising six distinct RTS can include the following RTS: Frt 1, Frt 2, Frt 3, Frt 4, Frt 5, Frt 6. In some embodiments, the cell comprising at least four distinct RTS can include the following RTS: Frt m5, Frt, Frt m14, Frt m15.

As referred to herein, the term “landing pad” refers to a nucleic acid sequence comprising a first recombination target site chromosomally-integrated into a host cell. In some embodiments, a landing site comprises two or more recombination target sites chromosomally-integrated into a host cell. In some embodiments, the cell comprises 1, 2, 3, 4, 5, 6, 7, or 8 landing pads. In some embodiments, the cell comprises 1, 2, or 3 landing pads. In some embodiments, the cell comprises 4 landing pads. In some embodiments, landing pads are integrated at up to 1, 2, 3, 4, 5, 6, 7, or 8 distinct chromosomal loci. In some embodiments, landing pads are integrated at up to 1, 2, or 3 distinct chromosomal loci. In some embodiments, landing pads are integrated at 4 distinct chromosomal loci.

In some embodiments, the disclosure describes how by expressing various proteins, e.g. DtE proteins, from various loci, it is possible to achieve the desired expression of the protein. In some embodiments, the DtE protein is expressed from the NL1 locus, e.g., on the CHO chromosome. In some embodiments, the DtE protein is expressed from the NL2 locus, e.g., on the CHO chromosome.

In some embodiments, the cell comprises two distinct RTS. In some embodiments, the two distinct RTS are chromosomally-integrated within the NL1 locus. In some embodiments, the two distinct RTS are chromosomally-integrated within the NL2 locus. In some embodiments, the cell comprises four distinct RTS. In some embodiments, the four distinct RTS are chromosomally-integrated on the same locus. In some embodiments, two distinct RTS are chromosomally-integrated within the NL1 locus or the NL2 locus, and two distinct RTS are chromosomally-integrated on a separate locus. In some embodiments, the separate locus is the Fer1L4 locus. In some embodiments, two distinct RTS are chromosomally-integrated within the NL1 locus, and two distinct RTS are chromosomally-integrated within the NL2 locus. In some embodiments, the cell comprises six distinct RTS. In some embodiments, at least four distinct RTS are chromosomally-integrated on the same locus. In some embodiments, at least two distinct RTS are chromosomally-integrated within the NL1 locus or the NL2 locus, and at least two distinct RTS are chromosomally-integrated on a separate locus. In some embodiments, the separate locus is the Fer1L4 locus. In some embodiments, at least two distinct RTS are chromosomally-integrated within the NL1 locus, and at least two distinct RTS are chromosomally-integrated within the NL2 locus. In some embodiments, at least one of the RTS is an frt site, a lox site, a rox site, or an att site. In some embodiments, at least one of the RTS is selected from among SEQ ID NOS.: 1-30.

Various cells can be used according to the present disclosure. In some embodiments, the cell is a mouse cell, a human cell, a Chinese hamster ovary (CHO) cell, a CHO-K1 cell, a CHO-DXB11 cell, a CHO-DG44 cell, a CHOK1SV™ cell including all variants (e.g. CHOK1SV™ POTELLIGENT®, Lonza, Slough, UK), a CHOK1SV GS-KO™ (glutamine synthetase knockout) cell including all variants, a HEK293 cell including adherent and suspension-adapted variants, a HeLa cell, or a HT1080 cell. In some embodiments, the mammalian cell comprises at least two distinct RTS, wherein the RTS are chromosomally-integrated within the NL1 locus, the NL2 locus or the Fer1L4 locus. In some embodiments, the mammalian cell comprises at least four distinct RTS wherein two distinct RTS are chromosomally-integrated within the NL1 locus or the NL2 locus and wherein two distinct RTS are chromosomally-integrated within a separate locus. In some embodiments, the separate locus is the Fer1L4 locus. In some embodiments, the mammalian cell comprises at least four distinct RTS, wherein two distinct RTS are chromosomally-integrated within the NL1 locus and two distinct RTs are chromosomally-integrated within the NL2 locus. In some embodiments, the mammalian cell comprises at least four distinct RTS wherein four distinct RTS are chromosomally-integrated within the NL1 locus or the NL2 locus. In some embodiments, the mammalian cell comprises at least six distinct RTS, where two distinct RTS are chromosomally-integrated within the NL1 locus, two distinct RTS are chromosomally-integrated within the NL2 locus, and two distinct RTS are chromosomally-integrated at a separate locus. In some embodiments, the separate locus is the Fer1L4 locus. In some embodiments, the mammalian cell comprises at least six distinct RTS, wherein six distinct RTS are integrated within the NL1 locus or the NL2 locus. In some embodiments, the mammalian cell comprises at least six distinct RTS, wherein four distinct RTS are integrated at the NL1 locus or the NL2 locus and wherein two distinct RTS are integrated at a separate locus. In some embodiments, the second locus is the Fer1L4 locus.

In some embodiments, the cell comprises a first gene of interest, wherein the first gene of interest is chromosomally-integrated.

As referred to herein, the term “gene of interest” or “GOI” is used to describe a heterologous gene. As referred to herein, the term “heterologous gene” or “HG” as it relates to nucleic acid sequences such as a coding sequence or a control sequence, denotes a nucleic acid sequence, e.g. a gene, that is not normally joined together, and/or are not normally associated with a particular cell. In some embodiments, a heterologous gene is a construct where the coding sequence itself is not found in nature (e.g., synthetic sequences having codons different from the native gene). Allelic variation or naturally occurring mutational events do not give rise to heterologous DNA, as used herein.

In some embodiments, the gene of interest comprises a reporter gene, a selection gene, a gene of therapeutic interest, an ancillary gene or a combination thereof.

As referred to herein, a “reporter gene” is a gene whose expression confers a phenotype upon a cell that can be easily identified and measured. In some embodiments, the reporter gene comprises a fluorescent protein gene. In some embodiments, the reporter gene comprises a selection gene.

As referred to herein, the term “selection gene” refers to the use of a gene which encodes an enzymatic activity that confers the ability to grow in medium lacking what would otherwise be an essential nutrient; in addition, a selection gene may confer resistance to an antibiotic or drug upon the cell in which the selection gene is expressed. A selection gene may be used to confer a particular phenotype upon a host cell. When a host cell must express a selection gene to grow in selective medium, the gene is said to be a positive selection gene. Selection gene can also be used to select against host cells containing a particular gene; selection genes used in this manner are referred to as negative selection genes.

As referred to herein, the terms “gene of therapeutic interest” refers to any functionally relevant nucleotide sequence. Thus, the gene of therapeutic interest of the present disclosure can comprise any desired gene that encodes a protein the expression of which is desired the preparation of a therapeutic recombinant protein. Representative (non-limiting) examples of suitable genes of therapeutic interest include monoclonal antibodies, bi-specific monoclonal antibodies, or antibody drug conjugates [include blood clotting factors, well expressed mAbs where protein expression is limited at transcription, hormones such as EPO, immune-fusion proteins (Fc fusions), tri-specific mAbs].

As referred to herein, the terms “ancillary gene” or “helper gene” are used interchangeable to refer to a first gene that aids in the expression of a second gene or that aids in the stabilization, folding, or post translational modification of the product of the second gene or that creates a cellular environment that promotes the production of the product of the second gene. In some embodiments, the second gene is a gene encoding a DtE protein. In some embodiments, the ancillary gene encodes RNA. In some embodiments, the ancillary gene encodes an mRNA, a tRNA, or a miRNA. In some embodiments, the ancillary gene encodes a transcription factor, a chaperone, a chaperonin, a synthetase, an oxidase, a reductase, a glycotransferase, a protease, a kinase, a phosphatase, an acetyl transferase, a lipase, or an alkylase.

In some embodiments, the first gene of interest comprises a reporter gene, a selection gene, a gene of therapeutic interest, an ancillary gene, or a combination thereof. In some embodiments, the gene of therapeutic interest comprises a gene encoding a well expressed therapeutic protein at a desired copy number. In some embodiments, the gene encoding a well expressed therapeutic protein is at a copy number of 2 copies, of 3 copies, of 4 copies, of 5 copies, of 6 copies, of 7 copies, of 8 copies, of 9 copies, or of 10 copies. In some embodiments, the gene of therapeutic interest comprises a gene encoding a DtE protein.

As referred to herein, the term a “difficult to express protein” refers to a protein for which production is difficult. In some embodiments, product of the DtE protein can be difficult because protein expression must be highly regulated. In some embodiments, the DtE protein is difficult to recover from the host cell. In some embodiments, the DtE protein is a protein that is prone to mis-folding. In some embodiments, the DtE protein is a protein that is prone to clipping. In some embodiments, the DtE protein is a protein that is prone to degradation. In some embodiments, the DtE protein is a protein that is prone to aggregation. In some embodiments, the DtE protein is a protein that is poorly soluble. In some embodiments, a DtE protein is a membrane bound protein. In some embodiments, the DtE protein is difficult to purify. In some embodiments, a DtE protein is cytotoxic. In some embodiments, the DtE protein comprises multiple polypeptide chains, e.g. 2, 3 or 4 polypeptide chains. In some embodiments, the multiple polypeptide chains of the DtE protein form a homo-oligomer to produce the DtE protein. In some embodiments, the multiple polypeptide chains of the DtE protein form a hetero-oligomer to produce the DtE protein. In some embodiments, the homo-oligomer or the hetero-oligomer is formed through covalent interactions, non-covalent interactions, or a combination thereof. In some embodiments, the DtE protein comprises a protein for which the expression of an ancillary gene is required to produce the DtE protein. In some embodiments, the DtE protein is a protein for which a post-translational modification is required to produce the DtE protein. In some embodiments, the DtE protein is a protein for which expression using standard techniques known to one of the art of molecular biology, results in a product protein with variable characteristics. In some embodiments, the DtE protein is a fusion protein.

For example, in some embodiments, the disclosure describes how expressing DtE proteins from NL1 and/or NL2 locus that DtE proteins can be obtained in more than 2 g/L protein production titers. In some embodiments, the expression of a DtE protein at the NL1 locus yields more than 2 g/L of the DtE protein. In some embodiments, the expression of a DtE protein at the NL2 locus yields more than 2 g/L of the DtE protein. In some embodiments, the DtE protein is a protein for which expression using standard techniques known to one of the art of molecular biology, results in a product protein titer less than 2 g/L.

In some embodiments, the DtE protein is a monoclonal antibody. In some embodiments, the DtE protein is a bi-specific monoclonal antibody. In some embodiments, the DtE protein is a tri-specific monoclonal antibody. In some embodiments, the DtE protein is an Fc-fusion protein. As referred to herein, the term “Fc-fusion protein” refers to a fusion protein wherein the Fc domain of an immunoglobulin is operably linked to a second peptide. In some embodiments, the DtE protein is an enzyme. In some embodiments, the DtE protein is a membrane receptor. In some embodiments, the DtE protein is a bi-specific T-cell engager (BITE® Micromet AG, Munich, Germany).

In some embodiments, the DtE protein is selected from the group consisting of an Fc-fusion protein, an enzyme, a membrane receptor, or a monoclonal antibody. In some embodiments, the monoclonal antibody is a bi-specific monoclonal antibody or a tri-specific monoclonal antibody.

In some embodiments, the DtE protein is encoded on one or more genes of interest. In some embodiments, the first gene of interest is located between two of the RTS.

As referred to herein, the term “located between two of the RTS” refers to a gene located between two of the RTS, i.e., with one of the RTS located 5′ of the gene and a different RTS located 3′ of the gene. In some embodiments, the RTS are located directly adjacent to the gene located between them. In some embodiments, the RTS are located at a defined distance from the gene located between them. In some embodiments, the RTS are directional sequences. In some embodiments, the RTS 5′ and 3′ of the gene located between them are directly oriented (i.e. they are oriented in the same direction). In some embodiments, the RTS 5′ and 3′ of the gene located between them are inversely oriented (i.e. they are oriented in opposite directions).

In some embodiments, the first gene of interest is located within the NL1 locus. In some embodiments, the cell comprises a second gene of interest, wherein the second gene of interest is chromosomally-integrated. In some embodiments, the second gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the DtE protein is selected from the group consisting of a Fc-fusion protein, an enzyme, a membrane receptor, a bi-specific T-cell engager (BITE®), or a monoclonal antibody. In some embodiments, the second gene of interest is located between two of the RTS. In some embodiments, the second gene of interest is located within the NL1 locus or the NL2 locus. In some embodiments, the first gene of interest is located within the NL1 locus, and the second gene of interest is located within the NL2 locus. In some embodiments, the cell comprises a third gene of interest, wherein the third gene of interest is chromosomally-integrated. In some embodiments, the third gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the third gene of interest is located between two of the RTS. In some embodiments, the third gene of interest is located within the NL1 locus or the NL2 locus. In some embodiments, the third gene of interest is located with a locus distinct from the NL1 locus and the NL2 locus. In some embodiments, the first gene of interest, the second gene of interest, and the third gene of interest are within three separate loci. In some embodiments, at least one of the first genes of interest, the second gene of interest, and the third gene of interest is within the NL1 locus, and at least one of the first gene of interest, the second gene of interest, and the third gene of interest is within the NL2 locus.

In some embodiments, the cell comprises a site-specific recombinase gene. In some embodiments, the site-specific recombinase gene is chromosomally-integrated.

In some embodiments, the present disclosure provides a mammalian cell comprising (a) at least four distinct RTS, wherein the cell comprises at least two distinct RTS are chromosomally-integrated within the NL1 locus or NL2 locus; (b) a first gene of interest is integrated between the at least two RTS of (a), wherein the first gene of interest comprises a reporter gene, a gene encoding a DtE protein, an ancillary gene or a combination thereof, and (c) a second gene of interest is integrated within a second chromosomal locus distinct from the locus of (a), wherein the second gene of interest comprises a reporter gene, a gene encoding a DtE protein, an ancillary gene or a combination thereof.

In some embodiments, the present disclosure provides a mammalian cell comprising (a) at least four distinct RTS, wherein the cell comprises at least two distinct RTS are chromosomally-integrated within the Fer1L4 locus; (b) at least two distinct RTS are chromosomally-integrated within the NL1 locus or the NL2 locus; (c) a first gene of interest is chromosomally-integrated within the Fer1L4 locus, wherein the first gene of interest comprises a reporter gene, a gene encoding a DtE protein, an ancillary gene or a combination thereof; and (d) a second gene of interest is chromosomally-integrated within the within the NL1 locus or NL2 locus of (b), wherein the second gene of interest comprises a reporter gene, a gene encoding a DtE protein, an ancillary gene or a combination thereof.

In some embodiments, the present disclosure provides a mammalian cell comprising at least six distinct RTS, wherein the cell comprises (a) at least two distinct RTS and a first gene of interest are chromosomally-integrated within the Fer1L4 locus; (b) at least two distinct RTS and a second gene of interest are chromosomally-integrated within the NL1 locus; and (c) at least two distinct RTS and a third gene of interest are chromosomally-integrated within the NL2 locus.

In some embodiments, the present disclosure provides a method for producing a recombinant protein producer cell comprising (a) providing a cell that comprises at least four distinct RTS and a gene encoding a site-specific recombinase, wherein at least two distinct RTS are chromosomally-integrated within the NL1 locus and at least two distinct RTS are chromosomally-integrated within the NL2 locus; (b) transfecting the cell of (a) with a first vector comprising an exchangeable cassette encoding a first gene of interest and a second vector comprising an exchangeable cassette encoding a second gene of interest; (c) integrating the first exchangeable cassette within the NL1 locus and the second exchangeable cassette within the NL2 locus; and (d) selecting a recombinant protein producer cell comprising the first exchangeable cassette and the second exchangeable cassette integrated into the chromosome.

“Transfection” as used herein means the introduction of an exogenous nucleic acid molecule, including a vector, into a cell. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to as “recombinant,” “transformed,” or “transgenic” organisms. A number of transfection techniques are generally known in the art. See, e.g., Graham et al., Virology, 52:456 (1973); Sambrook et al., Molecular Cloning, a laboratory manual, Cold Spring Harbor Laboratories, New York (1989); Davis et al., Basic Methods in Molecular Biology, Elsevier (1986); and Chu et al., Gene 13:197 (1981). Such techniques can be used to introduce one or more exogenous DNA moieties into suitable host cells.

The term “matching” in reference to two RTS sequences refers to two sequences that have the ability to be bound by a recombinase and to affect a site-specific recombination between the two sequences. In some embodiments, an RTS of an exchangeable cassette matching an RTS of the cell refers to the RTS of the cassette having a sequence substantially identical to the RTS of the cell. In some embodiments, the exchangeable cassette contains a sequence substantially identical to one or two of the RTS chromosomally-integrated into the host cell genome.

In some embodiments, the term integrating refers to the integration, e.g. insertion, of the exchangeable cassette into the chromosome. In some embodiments, integration is mediated by a site-specific recombinase. In some embodiments, the inventors find that the use of SSI eliminates the need to clone cells from those transfected, as the cells are homogenous in their genetic composition.

In some embodiments, the term “selecting” refers to identifying cells containing a chromosomally-integrated marker. In some embodiments, selection is through the detection of the presence of a marker using methods known to those skilled in the art. In some embodiments, selection is through the detection of the absence of a marker using methods known to those skilled in the art.

In some embodiments, the first gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the DtE protein consists of a Fc-fusion protein, an enzyme, a membrane receptor, a bi-specific T-cell engager (BITE®), or a monoclonal antibody. In some embodiments, the monoclonal antibody is a bi-specific monoclonal antibody or a tri-specific monoclonal antibody. In some embodiments, the first gene of interest is located between two of the RTS. In some embodiments, the second gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the second gene of interest is located between two of the RTS.

In some embodiments, the present disclosure provides a method for producing a recombinant protein producer cell comprising (a) providing a cell that comprises at least at least four distinct RTS and a gene encoding a site-specific recombinase, wherein at least two distinct RTS are chromosomally-integrated within the Fer1L4 locus, and at least two distinct RTS are chromosomally-integrated within the NL1 locus or the NL2 locus; (b) transfecting the cell of (a) with a first vector comprising an exchangeable cassette encoding a first gene of interest and a second vector comprising an exchangeable cassette encoding a second gene of interest; (c) integrating the first exchangeable cassette within the Fer1L4 locus and the second exchangeable cassette within the NL1 locus or the NL2 locus; and (d) selecting a recombinant protein producer cell comprising the first exchangeable cassette and the second exchangeable cassette integrated into the chromosome. In some embodiments, the first gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the DtE protein consists of a Fc-fusion protein, an enzyme, a membrane receptor, a bi-specific T-cell engager (BITE®), or a monoclonal antibody. In some embodiments, the monoclonal antibody is a bi-specific monoclonal antibody or a tri-specific monoclonal antibody. In some embodiments, the first gene of interest is located between two of the RTS. In some embodiments, the second gene of interest comprises a reporter gene, a selection gene, a gene of therapeutic interest, an ancillary gene, or a combination thereof. In some embodiments, the gene of therapeutic interest comprises a gene encoding a DtE protein. In some embodiments, the second gene of interest is located between two of the RTS.

In some embodiments, the present disclosure provides a method for producing a recombinant protein producer cell comprising (a) providing a cell that comprises at least at least six distinct RTS and a gene encoding a site-specific recombinase, wherein at least two distinct RTS are chromosomally-integrated within the Fer1L4 locus, and at least two distinct RTS are chromosomally-integrated within the NL1 locus, and at least two distinct RTS are chromosomally-integrated within the NL2 locus; (b) transfecting the cell of (a) with a first vector comprising an exchangeable cassette encoding a first gene of interest, a second vector comprising an exchangeable cassette encoding a second gene of interest, and a third vector comprising an exchangeable cassette encoding a third gene of interest; (c) integrating the first exchangeable cassette within the Fer1L4 locus, the second exchangeable cassette within the NL1 locus, and the third exchangeable cassette within the NL2 locus; and (d) selecting a recombinant protein producer cell comprising the first exchangeable cassette, the second exchangeable cassette and the third exchangeable cassette integrated into the chromosome.

In some embodiments, the inventors have found that the use of SSI to prepare rP expression cells ensures the pool of rP expression cells is homogenous in its genetic makeup. In some embodiments, the inventors have found that the use of SSI to prepare rP expression cells ensures the pool of rP expression cells is homogenous in its efficiency. In some embodiments, the inventors have found that the use of SSI to prepare rP expression cells ensures the pool of producer cells is homogenous in the ratio of a first helper gene to a second helper gene. In some embodiments, the inventors have found that the use of SSI to prepare rP expression cells ensures the pool of producer cells is homogenous in the ratio of helper genes to genes of therapeutic interest. In some embodiments, the inventors have found that the use of SSI to prepare rP expression cells ensures more consistent rP product quality.

The cell lines described herein, including prokaryotic and/or eukaryotic cell lines, can be cultured using any suitable device, facility and methods described herein. Further, in embodiments, the devices, facilities and methods are suitable for culturing suspension cells or anchorage-dependent (adherent) cells and are suitable for production operations configured for production of pharmaceutical and biopharmaceutical products-such as polypeptide products, nucleic acid products (for example DNA or RNA), or mammalian or microbial cells and/or viruses such as those used in cellular and/or viral and microbiota therapies.

In some embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, devices, facilities and methods allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesized by the eukaryotic cells in a large-scale manner. In some embodiments, also disclosed are the use of microbial organisms and spores thereof utilized in microbiota therapeutics. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor or bioreactor including but not limited to stirred tank, airlift, fiber, microfiber, hollow fiber, ceramic matrix, fluidized bed, fixed bed, and/or spouted bed bioreactors. As used herein, “reactor” or “bioreactor” can include a fermenter or fermentation unit, or any other reaction vessel and the term “reactor” is used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (e.g., NS0 or SP2/0 cell lines), Chinese hamster ovary (CHO) cell lines, HT1080, H9, HepG2, MCF7, MDBK Jurkat, NIH3T3, PC12, BHK (baby hamster kidney cell), VERO, YB2/0, Y0, C127, L cell, COS, e.g., COS1 and COS7, QC1-3, HEK-293, VERO, PER.C6, HeLa, EB1, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHOK1SV™ GS knockout cell (CHOK1SV GS-KO™). The CHO FUT8 knockout cell is, for example, the CHOK1SV™ POTELLIGENT® (Lonza Biologics, Inc.). Eukaryotic cells can also be avian cells, cell lines or cell strains, such as for example, EBX® cells, EB14, EB24, EB26, EB66, or EBvl3.

In some embodiments, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In one embodiment, the cell is a differentiated form of any of the cells described herein. In one embodiment, the cell is a cell derived from any primary cell in culture. In some embodiments, the cells are not derived from stem cells. For example, in some embodiments, the cells are used in immunotherapies (e.g., lymphocytes) either extracted or isolated from individual patients or from established cell banks. In some embodiments, the cells can include genetically manipulated cells (i.e. CAR-T, etc.)

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic Kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). Example hepatocytes are commercially available from Triangle Research Labs, LLC, 6 Davis Drive Research Triangle Park, N.C., USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii), the Geotrichum genus (e.g. Geotrichum fermentans), Hansenula polymorpha, Yarrowia lipolytica, or Schizosaccharomyces pombe. Preferred is the species Pichia pastoris. Examples for Pichia pastoris strains are X33, GS115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, MIMIC™ Sf9, Sf21, HIGH FIVE™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell. In some embodiments, the prokaryotic cell is a Gram-positive cell such as Bacillus, Streptomyces Streptococcus, Staphylococcus or Lactobacillus. Bacillus that can be used is, e.g. the B. subtilis, B. amyloliquefaciens, B. licheniformis, B. natto, or B. megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

In one embodiment, the prokaryotic cell is a Gram-negative cell, such as Salmonella spp. or Escherichia coli, such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as for example BL-21 or BL21 (DE3), all of which are commercially available. Suitable host cells are commercially available, for example, from culture collections such as the DSMZ (Deutsche Sammlung von Mikroorganismen and Zellkulturen GmbH, Braunschweig, Germany) or the American Type Culture Collection (ATCC). In some embodiments, the cells include other microbiota utilized as therapeutic agents. These include microbiota present in the human microbiome belonging to the phyla Firmicutes, Bacteroidetes, Proteobacteria, Verrumicrobia, actinobacteria, fusobacteria and cyanobacteria. Microbiota can include both aerobic, strict anaerobic or facultative anaerobic and include cells or spores. Therapeutic Microbiota can also include genetically manipulated organisms and vectors utilized in their modification. Other microbiome-related therapeutic organisms can include: archaea, fungi and virus. See, e.g., The Human Microbiome Project Consortium. Nature 486, 207-214 (14 Jun. 2012); Weinstock, Nature, 489(7415): 250-256 (2012); Lloyd-Price, Genome Medicine 8:51 (2016).

In some embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 Daltons to greater than about 140,000 Daltons can be produced. In embodiments, these molecules can have a range of complexity and can include post-translational modifications including glycosylation.

In embodiments, the protein is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-la), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex’ recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In some embodiments, the polypeptide is adalimumab (HUMIRA), infliximab (REMICADE™), rituximab (RITUXAN™/MABTHERA™) etanercept (ENBREL™), bevacizumab (AVASTIN™), trastuzumab (HERCEPTIN™), pegrilgrastim (NEULASTA™), or any other suitable polypeptide including biosimilars and biobetters.

Other suitable polypeptides are those listed below in Table 6 and in Table 1 of US2016/0097074. One of skill in the art can appreciate that the disclosure of the present invention additional would encompass combinations of products and/or conjugates as described herein [(i.e. multi-proteins, modified proteins (conjugated to PEG, toxins, other active ingredients)].

TABLE 6

| Protein Product | Reference Listed Drug |
|---|---|
| interferon gamma-1b | ACTIMMUNE ® |
| alteplase; tissue plasminogen activator | ACTIVASE ®/CATHFLO ® |
| recombinant antihemophilic factor | ADVATE |
| human albumin | ALBUTEIN ® |
| Laronidase | ALDURAZYME ® |
| interferon alfa-N3, human leukocyte derived | ALFERON N ® |
| human antihemophilic factor | ALPHANATE ® |
| virus-filtered human coagulation factor IX | ALPHANINE ® SD |
| Alefacept; recombinant, dimeric fusion protein LFA3-Ig | AMEVIVE ® |
| Bivalirudin | ANGIOMAX ® |
| darbepoetin alfa | ARANESP ™ |
| Bevacizumab | AVASTIN ™ |
| interferon beta-1a; recombinant | AVONEX ® |
| coagulation factor IX | BENEFIX ™ |
| interferon beta-1b | BETASERON ® |
| Tositumomab | BEXXAR ® |
| antihemophilic factor | BIOCLATE ™ |
| human growth hormone | BIOTROPIN ™ |
| botulinum toxin type A | BOTOX ® |
| Alemtuzumab | CAMPATH ® |
| acritumomab; technetium-99 labeled | CEA-SCAN ® |
| alglucerase; modified form of beta-glucocerebrosidase | CEREDASE ® |
| imiglucerase; recombinant form of beta- | CEREZYME ® |
| glucocerebrosidase | |
| crotalidae polyvalent immune Fab, ovine | CROFAB ™ |
| Digoxin immune fab [ovine] | DIGIFAB ™ |
| Rasburicase | ELITEK ® |
| Etanercept | ENBREL ® |
| Epoietin alfa | EPOGEN ® |
| Cetuximab | ERBITUX ™ |
| Algasidase beta | FABRAZYME ® |
| Urofollitropin | FERINEX ™ |
| Follitropin beta | FOLLISTIM ™ |
| Teriparatide | FORTEO ® |
| Human somatropin | GENOTROPIN ® |
| Glucagon | GLUCAGEN ® |
| Follitropin alfa | GONAL-F ® |
| Antihemophillic factor | HELIXATE ® |
| Antihemophilic factor; Factor XIII | HEMOFIL |
| Adefovir dipivoxil | HEPSERA ™ |
| trastuzumab | HERCEPTIN ® |
| Insulin | HUMALOG ® |
| Antihemophilic factor/von Willebrand factor complex- | HUMATE-P ® |
| human | |
| Somatotropin | HUMATROPE ® |
| Adalimumab | HUMIRA ™ |
| Human insulin | HUMULIN ® |
| Recombinant human hyaluronidase | HYLENEX ™ |
| Interferon alfacon-1 | INFERGEN ® |
| Eptifibatide | INTEGRILLIN ™ |
| Alpha-interferon | INTRON A ® |
| Palifermin | KEPIVANCE |
| Anakinra | KINERET ™ |
| Antihemophilic factor | KOGENATE ®FS |
| Insulin glargine | LANTUS ® |
| Granulocyte macrophage colony-simulating factor | LEUKINE ®/LEUKINE ®LIQUID |
| Lutropin alfa for injection | LUVERIS |
| OspA lipoprotein | LYMERIX ™ |
| Ranibizumab | LUCENTIS ® |
| Gemtuzumab ozogamicin | MYLOTARG ™ |
| Galsulfase | NAGLAZYME ™ |
| Nesiritide | NATRECOR ® |
| Pegfilgrastim | NEULASTA ™ |
| Oprelvekin | NEUMEGA ® |
| Filgrastim | NEUPOGEN ® |
| Fanolesomab | NEUTROSPEC ™ (FORMERLY |
| | LEUTECH ®) |
| Somatropin [rDNA] | NORDITROPIN ®/NORDITROPIN |
| | NORDIFLEX ® |
| Mitoxantrone | NOVANTRONE ® |
| Insulin; zinc suspension | NOVOLIN L ® |
| Insulin; isophane suspension | NO VOLIN N ® |
| Insulin, regular | NOVOLIN R ® |
| Insulin | NOVOLIN ® |
| Coagulation factor VIIa | NOVOSEVEN ® |
| Somatropin | NUTROPIN ® |
| Immunoglobulin intravenous | OCTAGAM ® |
| PEG-L-asparaginase | ONCASPAR ® |
| Abatacept, fully human soluable fusion protein | ORENCIA ™ |
| Muromomab-CD3 | ORTHOCLONE OKT3 ® |
| High-molecular weight hyaluronan | ORTHOVISC ® |
| Human chorionic gonadotropin | OVIDREL ® |
| Live attenuated Bacillus Calmette-Guerin | PACIS ® |
| Peginterferon alfa-2a | PEGASYS ® |
| Pegylated version of interferon alfa-2b | PEG-INTRON ™ |
| Abarelix (Injection suspension); gonadotropin-releasing | PLENAXIS ™ |
| hormone antagonist | |
| Epoietin alfa | PROCRIT ® |
| Aldesleukin | PROLEUKIN, IL-2 ® |
| Somatrem | PROTROPIN ® |
| Dornase alfa | PULMOZYME ® |
| Efalizumab; selective, reversible T-cell blocker | RAPTIVA ™ |
| Combination of ribavirin and alpha interferon | REBETRON ™ |
| Interferon beta 1a | REBIF ® |
| Antihemophilic factor | RECOMBINATE ® RAHF |
| Antihemophilic factor | REFACTO ® |
| Lepirudin | REFLUDAN ® |
| Infliximab | REMICADE ® |
| Abciximab | REOPRO ™ |
| Reteplase | RETAVASE ™ |
| Rituxima | RITUXAN ™ |
| Interferon alfa-2^{a} | ROFERON-A ® |
| Somatropin | SAIZEN ® |
| Synthetic porcine secretin | SECREFLO ™ |
| Basiliximab | SIMULECT ® |
| Eculizumab | SOLIRIS ® |
| Pegvisomant | SOMAVERT ® |
| Palivizumab; recombinantly produced, humanized mAb | SYNAGIS ™ |
| Thyrotropin alfa | THYROGEN ® |
| Tenecteplase | TNKASE ™ |
| Natalizumab | TYSABRI ® |
| Human immune globulin intravenous 5% and 10% | VENOGLOBULIN-S ® |
| solutions | |
| Interferon alfa-n1, lymphoblastoid | WELLFERON ® |
| Drotrecogin alfa | XIGRIS ™ |
| Omaluzumab; recombinant DNA-derived humanized | XOLAIR ® |
| monoclonal antibody targeting immunoglobulin-E | |
| Daclizumab | ZENAPAX ® |
| Ibritumomab tiuxetan | ZEVALIN ™ |
| Somatotropin | ZORBTIVE ™ (SEROSTIM ®) |

In embodiments, the polypeptide is a hormone, blood clotting/coagulation factor, cytokine/growth factor, antibody molecule, fusion protein, protein vaccine, or peptide as shown in Table 7.

TABLE 7
Exemplary Products

| Therapeutic | | |
|---|---|---|
| Product type | Product | Trade Name |
| Hormone | Erythropoietin, Epoein-α | Epogen, Procrit |
| | Darbepoetin-α | Aranesp |
| | Growth hormone (GH), | Genotropin, Humatrope, |
| | somatotropin | Norditropin, NovIVitropin, |
| | | Nutropin, Omnitrope, Protropin, |
| | | Siazen, Serostim, Valtropin |
| | Human follicle-stimulating hormone | Gonal-F, Follistim |
| | (FSH) | |
| | Human chorionic gonadotropin | Ovidrel, Luveris |
| | Lutropin-α | GlcaGen |
| | Glucagon | Geref |
| | Growth hormone releasing hormone | ChiRhoStim (human peptide), |
| | (GHRH) | SecreFlo (porcine peptide) |
| | Secretin | |
| | Thyroid stimulating hormone | Thyrogen |
| | (TSH), thyrotropin | |
| Blood Clotting/ | Factor VIIa | NovoSeven |
| Coagulation | Factor VIII | Bioclate, Helixate, Kogenate, |
| Factors | | Recombinate, ReFacto |
| | Factor IX | Benefix |
| | Antithrombin III (AT-III) | Thrombate III |
| | Protein C concentrate | Ceprotin |
| Cytokine/Growth | Type I alpha-interferon | Infergen |
| factor | Interferon-αn3 (IFNαn3) | Alferon N |
| | Interferon-β1a (rIFN- β) | Avonex, Rebif |
| | Interferon-β1b (rIFN- β) | Betaseron |
| | Interferon-γ1b (IFN γ) | Actimmune |
| | Aldesleukin (interleukin 2(IL2), | Proleukin |
| | epidermal theymocyte activating | |
| | factor; ETAF | |
| | Palifermin (keratinocyte growth | Kepivance |
| | factor; KGF) | |
| | Becaplemin (platelet-derived growth | Regranex |
| | factor; PDGF) | |
| | Anakinra (recombinant IL1 | Anril, Kineret |
| | antagonist) | |
| Antibody | Bevacizumab (VEGFA mAb) | Avastin, Erbitux |
| molecules | Cetuximab (EGFR mAb) | Vectibix |
| | Panitumumab (EGFR mAb) | Campath |
| | Alemtuzumab (CD52 mAb) | Rituxan |
| | rituximab (CD20 chimeric Ab) | Herceptin, Orencia |
| | Trastuzumab (HER2/Neu mAb) | Humira, Enbrel |
| | Abatacept (CTLA Ab/Fc fusion) | Remicade |
| | Adalimumab (TNFα mAb) | Amevive |
| | Etanercept (TNF receptor/Fc fusion) | Raptiva, Tysabri |
| | Infliximab (TNFα chimeric mAb) | Soliris, Orthoclone, OKT3 |
| | Alefacept (CD2 fusion protein) | |
| | Efalizumab (CD11a mAb) | |
| | Natalizumab (integrin α4 subunit | |
| | mAb) | |
| | Eculizumab (C5mAb) | |
| | Muromonab-CD3 | |
| Other: | Insulin | Humulin, Novolin |
| | Hepatitis B surface antigen | Engerix, Recombivax HB |
| | (HBsAg) | Gardasil |
| Fusion | HPV vaccine | LYMErix |
| proteins/Protein | OspA | Rhophylac |
| vaccines/Peptides | Anti-Rhesus(Rh) immunoglobulin G | Fuzeon |
| | Enfuvirtide | |
| | Spider silk, e.g., fibrion | QMONOS |

In embodiments, the protein is multispecific protein, e.g., a bispecific antibody as shown in Table 8.

TABLE 8
Bispecific Formats

| Name (other | | | | | |
|---|---|---|---|---|---|
| names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, EpCAM | Retargeting of T | Approved in | Malignant |
| (REMOVAB ®, | Triomab | | cells to tumor, Fc | EU | ascites in |
| Fresenius Biotech, | | | mediated effector | | EpCAM positive |
| Trion Pharma, | | | functions | | tumors |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius Biotech) | | | | | |
| Blinatumomab | BiTE | CD3, CD19 | Retargeting of T | Approved in | Precursor B-cell |
| (BLINCYTO ®, | | | cells to tumor | USA | ALL |
| AMG 103, MT | | | | Phase II and | ALL |
| 103, MEDI 538, | | | | III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, EpCAM | Retargeting of T | Phase I | Solid tumors |
| 110, MT110, | | | cells to tumor | | |
| Amgen) | | | | | |
| MEDI 565 (AMG | BiTE | CD3, CEA | Retargeting of T | Phase I | Gastrointestinal |
| 211, MedImmune, | | | cells to tumor | | adenocancinoma |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, Bayer; | | | cells to tumor | | |
| Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal cancer |
| (Macrogenics) | | | cells to tumor | | |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of | | |
| Therapeutics) | | | NK cells to tumor | | |
| | | | cells | | |
| AFM13 (Affimed | TandAb | CD30, CD16A | Retargeting of | Phase II | Hodgkin's |
| Therapeutics) | | | NK cells to tumor | | Lymphoma |
| | | | cells | | |
| GD2 (Barbara Ann | T cells | CD3, GD2 | Retargeting of T | Phase I/II | Neuroblastoma |
| Karmanos Cancer | preloaded | | cells to tumor | | and |
| Institute) | with BsAb | | | | osteosarcoma |
| pGD2 (Barbara | T cells | CD3, Her2 | Retargeting of T | Phase II | Metastatic breast |
| Ann Karmanos | preloaded | | cells to tumor | | cancer |
| Cancer Institute) | with BsAb | | | | |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR-armed | T cells | CD3, EGFR | Autologous | Phase I | Colon and |
| activated T-cells | preloaded | | activated T cells | | pancreatic |
| (Barbara Ann | with BsAb | | to EGFR-positive | | cancers |
| Karmanos Cancer | | | tumor | | |
| Institute) | | | | | |
| rM28 (University | Tandem | CD28, MAPG | Retargeting of T | Phase II | Metastatic |
| Hospital Tübingen) | scFv | | cells to tumor | | melanoma |
| IMCgp100 | ImmTAC | CD3, peptide MHC | Retargeting of T | Phase I/II | Metastatic |
| (Immunocore) | | | cells to tumor | | melanoma |
| DT2219ARL | 2 scFv | CD19, CD22 | Targeting of | Phase I | B cell leukemia |
| (NCI, University | linked to | | protein toxin to | | or lymphoma |
| of Minnesota) | diphtheria | | tumor | | |
| | toxin | | | | |
| XmAb5871 | BsAb | CD19, CD32b | | | |
| (Xencor) | | | | | |
| NI-1701 | BsAb | CD47, CD19 | | | |
| (NovImmune) | | | | | |
| MM-111 | BsAb | ErbB2, ErbB3 | | | |
| (Merrimack) | | | | | |
| MM-141 | BsAb | IGF-1R, ErbB3 | | | |
| (Merrimack) | | | | | |
| NA (Merus) | BsAb | HER2, HER3 | | | |
| NA (Merus) | BsAb | CD3, CLEC12A | | | |
| NA (Merus) | BsAb | EGFR, HER3 | | | |
| NA (Merus) | BsAb | PD1, undisclosed | | | |
| NA (Merus) | BsAb | CD3, undisclosed | | | |
| Duligotuzumab | DAF | EGFR, HER3 | Blockade of 2 | Phase I and II | Head and neck |
| (MEHD7945A, | | | receptors, ADCC | Phase II | cancer |
| Genentech, Roche) | | | | | Colorectal cancer |
| LY3164530 (Eli | Not | EGFR, MET | Blockade of 2 | Phase I | Advanced or |
| Lily) | disclosed | | receptors | | metastatic cancer |
| MM-111 | HSA body | HER2, HER3 | Blockade of 2 | Phase II | Gastric and |
| (Merrimack | | | receptors | Phase I | esophageal |
| Pharmaceuticals) | | | | | cancers |
| | | | | | Breast cancer |
| MM-141, | IgG-scFv | IGF-1R, HER3 | Blockade of 2 | Phase I | Advanced solid |
| (Merrimack | | | receptors | | tumors |
| Pharmaceuticals) | | | | | |
| RG7221 | CrossMab | Ang2, VEGFA | Blockade of 2 | Phase I | Solid tumors |
| (RO5520985, | | | proangiogenics | | |
| Roche) | | | | | |
| RG7716 (Roche) | CrossMab | Ang2, VEGFA | Blockade of 2 | Phase I | Wet AMD |
| | | | proangiogenics | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1β | Blockade of 2 | Phase II | Osteoarthritis |
| (AbbVie) | | | proinflammatory | | |
| | | | cytokines | | |
| ABT-122 | DVD-Ig | TNF, IL-17A | Blockade of 2 | Phase II | Rheumatoid |
| (AbbVie) | | | proinflammatory | | arthritis |
| | | | cytokines | | |
| COVA322 | IgG- | TNF, IL17A | Blockade of 2 | Phase I/II | Plaque psoriasis |
| | fynomer | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem IgG | | cytokines | | fibrosis |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, HSA | Blockade of | Phase II | Rheumatoid |
| (ATN103, Ablynx) | | | proinflammatory | | arthritis |
| | | | cytokine, binds to | | |
| | | | HSA to increase | | |
| | | | half-life | | |
| ALX-0761 (Merck | Nanobody | IL-17A/F, HSA | Blockade of 2 | Phase I | (Healthy |
| Serono, Ablynx) | | | proinflammatory | | volunteers) |
| | | | cytokines, binds | | |
| | | | to HSA to | | |
| | | | increase half-life | | |
| ALX-0061 | Nanobody | IL-6R, HSA | Blockade of | Phase I/II | Rheumatoid |
| (AbbVie, Ablynx; | | | proinflammatory | | arthritis |
| | | | cytokine, binds to | | |
| | | | HSA to increase | | |
| | | | half-life | | |
| ALX-0141 | Nanobody | RANKL, HSA | Blockade of bone | Phase I | Postmenopausal |
| (Ablynx, | | | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

### EXAMPLES

### Example 1: Identification of High Expression and Stable Loci

Four hundred GS-CHOK1SV™ clonal cell lines (Lonza, Basel, Switzerland) producing monoclonal antibody (mAb), constructed using random integration, were screened for those which met the following criteria: high qmAb (>1.25 pg/cell·h), stable productivity (>70 generations) and suitable growth (IVCC>1500×10⁶ cells/mL·h, μ˜0.03 h⁻¹). The majority of these cell lines were derived from two published Lonza projects (Porter et al., Cell Culture and Tissue Engineering 26:1455-1464 (2010) and Povey et al., J Biotechnol 184-93 (2014) and the process for the construction of GS-CHOK1SV™ clonal cell lines is described in detail in Porter et al., 2010. In short, a PvuI-linearized Lonza glutamine synthase (GS) expression vector encoding a mAb was introduced into the host cell line, CHOK1SV™, using standard electroporation methods. The transfection mixture was distributed across eighty 96-well plates. The following day, fresh medium was added to the cell suspension in the plates; the methionine sulfoximine (MSX) concentration in the medium was such that the final MSX concentration in each well was 50 μM. As the biomass of colonies was increased, cell lines were cultured in static 24-well plates and then in static 25 cm² T-flasks. Suspension cultures were initiated from confluent 25 cm² T-flasks and expanded for 10 L fed-batch bioreactor culture and cryopreservation.

The lowest generation number ampoule for these twenty cell lines was cultured to mid-exponential culture phase for analysis of integrated vector copy number using qPCR (primer/probe sets for beta lactamase gene). Five cell lines (964E7, 952C8, 2A6, E22 and E14) identified as meeting copy number criteria (≤5 beta lactamase copies per genome) were sampled for flanking sequence identification.

Genomic DNA (gDNA) extracts were prepared with cells derived from the aforementioned 5 cell lines and were subjected to sequence capture analysis. NimbleGen SeqCap Target Enrichment (Roche NimbleGen, Inc., Madison, USA) was completed on fragmented gDNA derived from recombinant cell lines using baits designed for regions within the glutamine synthase (GS) expression vector bearing mAb genes. Target enriched pools were eluted and sequenced by Illumina MISEQ® (Next Gen Sequencing, Illumina, San Diego, Calif., USA). The same five cell lines were analyzed by whole genome re-sequencing (WGRS) (BGI, Tai Po, Hong Kong) and two selected cell lines (964E7 and 952C8) were analysis using targeted locus amplification (TLA) (Cergentis B.V., Netherlands) to validate the results of targeted sequencing and WGRS.

Bioinformatic analysis of sequencing reads was conducted. Capture sequencing data were mapped to genome and vector sequences. Split reads (half from genome, half from vector) were used to identify potential integration sites and the break point. In addition, read pairs that suggest vector insertion (one end on genome, one end on vector) were also identified to provide support evidence for the integration sites. A list of potential integration sites was identified from sequence capture data, and the sites were ranked by the supporting evidence. WGRS data were mapped to all potential integration sites to provide additional support evidence. Reads from WGRS that map across a break point, as well as read pairs that cover the break points were counted as positive supports. For each integration site, left (L) and right (R) were used to refer to the locations of the genomic sequence (always 5′ to 3′ according to Cricetulus griseus scaffold and contig sequences). At left or right sides the vector was inserted in the forward (F) or reverse direction (R). Data relating to cell lines 964E7 and 952C8 were later by targeted sequencing with proximity ligation at Cergentis (Utrecht, NL) see, e.g. de Vree et al., Nat Biotechnol. 32:1019-25 (2014).

Integration sites were validated by PCR amplification across predicted genome: vector boundaries in all five cell lines. Table 9 summarizes these integration site findings, along-side the beta-lactamase gene copy number, productivity and growth data for these recombinant cell lines. A total of six sites (New Loci 1-6 (NL1-6)) were confirmed by PCR in the five cells lines, three which were confirmed by PCR across both genome vector boundaries (NL1, NL2 and NL4) and three which were confirmed at only one of the boundaries (NL3, NL5 and NL6).

TABLE 9

| Cell Line Data | | | | | | | | Genome | |
|---|---|---|---|---|---|---|---|---|---|
| | Copies β- | | qP | | | | | Vector | |
| Cell | Lactamase/ | | (pg/cell. | | Titer | IVCC | Stability | Boundaries | Scaffold |
| Line | genome | mAb | day) | Host | (g/L) | (10⁶ cells/h · mL) | (G) | Confirmed | Accession |
| 964E7 | 2.8 | H31K5 | 48 | CHOK1SV ™ | 7.6 | 3764 | 81G | Both flanks | gI351515650 |
| 952C8 | 3.0 | H31K5 | 47 | CHOK1SV ™ | 4.0 | 2039 | 69G | Both flanks | gI351517715 |
| 2A6 | 3.7 | cB72.3 | 23 | GSKO | 6.0 | 6122 | 70G | One flank | gI351516540 |
| E22 | 2.6 | cB72.3 | 23 | GSKO | 6.0 | 5109 | 70G | Both flanks | gI351516248 |
| E22 | | | | | | | | One flank | gI351517397 |
| E14 | 2.8 | cB72.3 | 19 | GSKO | 4.8 | 6015 | 70G | One flank | gI351516957 |

Loci NL1 and NL2 were progressed to SSI landing pad integration, as derivative cell lines 964E7 and 952C8 had similar integrated beta-lactamase copies to the other 3 cell lines, but achieved a higher specific productivity (47-48 pg/cell·day) suggesting these regions support higher recombinant gene expression. The selection of loci from recombinant cell lines generated in CHOK1SV™ derivative hosts and using a GS selection marker (see stage 2 for description of RMCE step) ensured that loci are compatible with a GS expression system based system. These loci have been shown to support stable recombinant gene expression (qP: 23-48 pg/cells·day) without negatively affecting process important to growth (IVCC: 2039 to 6015×10⁶ cells/h·mL).

### Example 2: Engineering of Landing Pads into Combinations of Selected Loci

Landing pads suitable for subsequent RMCE were integrated into the CHOK1SV GS-KO™ host cell line.

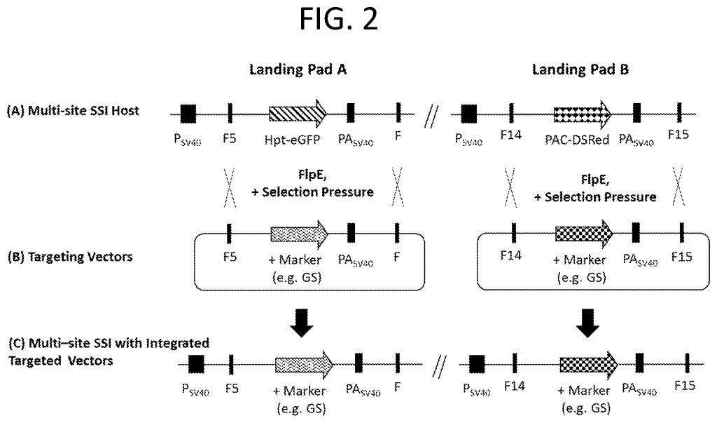

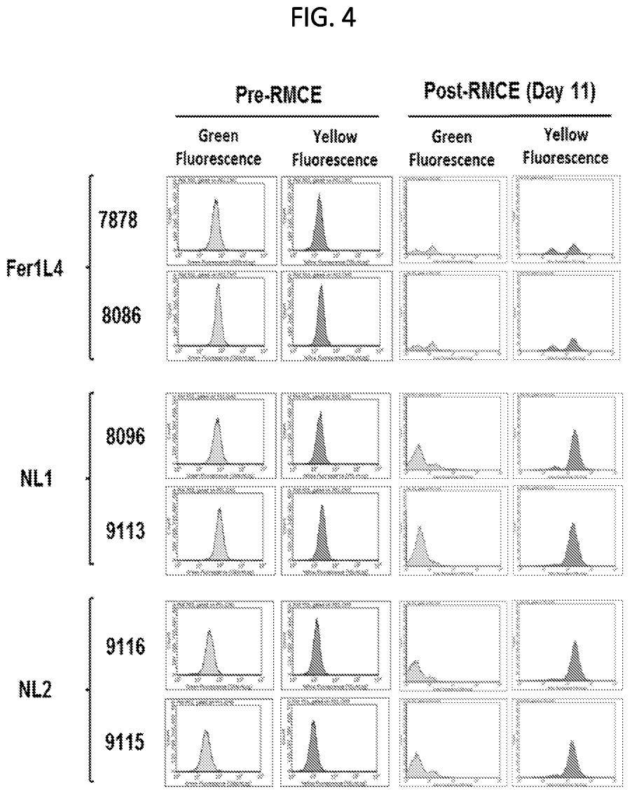

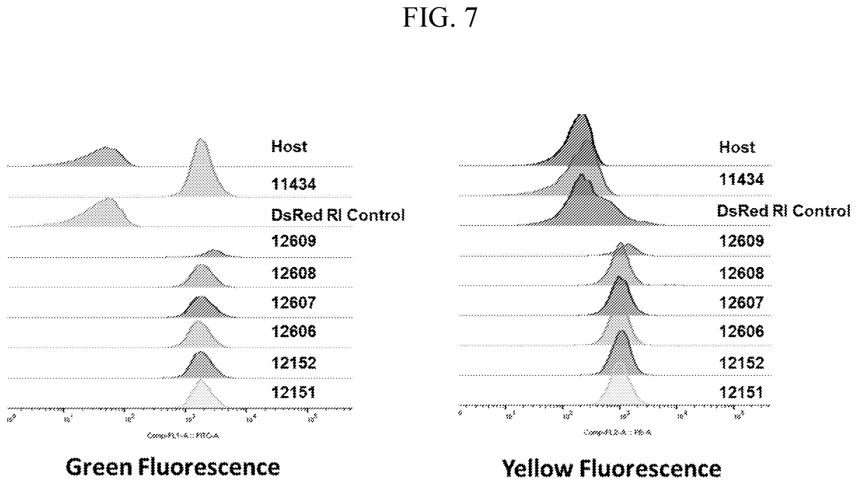

A landing pad (Landing Pad A: FIG. 2) was initially integrated separately into Fer1L4 (see, e.g., WO2013190032A1 and EP2711428A1) (FIG. 4: Clones 7878 and 8086, FIG. 7: Clone 11434), NL1 (FIG. 4: Clones 8096 and 9113) and NL2 (Clones 9116 and 9115) loci. Clone 11434 (landing pad in the Fer1L4 locus, Landing Pad A: FIG. 2A), was selected for engineering of the second landing pad at NL1 (Landing Pad B: FIG. 2A). This decision was made on the basis of the ability to complete RMCE when using with GS selection, stability of RFP expression in pools during repeated sub-culture and concentration of mAb secreted into the medium of fed-batch cultures at harvest. We have successfully demonstrated the general possibility that a landing pad of A or B type could go into any loci in Table 1. A total of 6 multisite SSI clones were generated (12151, 12152, 12606, 12607, 12608 and 12609). These 2-site hosts have a landing pad in Fer1L4 containing Hpt-eGFP fusion flanked by Frt F5 and wild-type Frt F RTS and a second landing pad in site 2 containing a PAC-DsRed fusion gene flanked by Frt F14 and Frt F15 RTS (landing pad in the NL1 loci, Landing Pad B: FIG. 2). The positioning of the Frt site between the SV40E promoter and selection marker enables it to be used in subsequent rounds of RMCE.

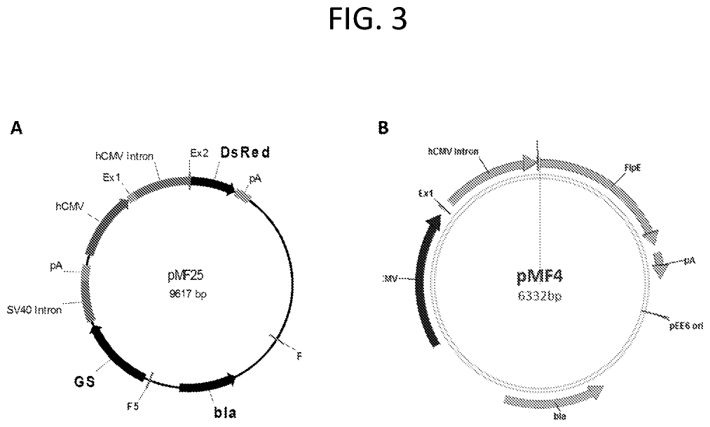

Targeting vectors designed for RMCE in the CHOK1SV GS-KO™ single and multi-landing pad hosts contained the GS cDNA arranged immediately to the 3′ of a Frt site compatible with the destination landing pad (FIG. 2B). The remainder of the vector contained transcription units for the GOI (e.g., mAb) followed by a Frt site compatible to the second Frt site in the landing pad. Targeting vector DNA (FIG. 3A) were co-transfected with a vector expressing FlpE recombinase (FIG. 3B) (at a plasmid molar ratio of 1:9, respectively). Transfected cells were incubated for 24 hours in the presence of 6 mM glutamine to allow transient expression of the FlpE recombinase. This transfectant pool was then washed and incubated in medium lacking glutamine. The viable cell concentration and culture viability were monitored throughout selection. Successful RMCE was marked by the loss of the Hpt-eGFP gene and replaced with GS gene in the targeting vector. A no-FlpE control was included in all transfections (transfection of targeting vector DNA without pMF4) to confirm any recovery is the result of transient FlpE recombinase expression (RMCE). Upon successful RMCE cells appeared dark under fluorescent microscope or with flow cytometry analysis. As the CHOK1SV GS-KO™ host lack a functional endogenous GS gene, those cells which had an on-target integration of GS were able to grow in the absence of glutamine and 12-14 days post-transfection culture viability was above 95%. As the GS cDNA in the targeting vector lacked a promoter, there was very low probability that off-target integration of the GS gene would result in sufficient GS protein amounts to enable growth in the absence of glutamine. Selection through inhibition of endogenous enzyme activity may have off target effects (e.g. MSX, see Feary et al., Biotechnol. Prog. (2016)) and therefore selection in the absence of a metabolite such as glutamine was favorable. This selection system was extremely stringent, with the percentage of non-exchanged cells (marked by the presence of GFP fluorescence) 14 days after transfection typically at around 2-5% and were easily removed by fluorescence-aided cell sorting. Where two sites are to be targeted by one vector, multiple Frt sites can be arranged as a concatemer simplifying the transfection.

### Example 3: Evaluating Fer1L4, NL1 and NL2 Loci Separately in the CHOK1SV GS-KO™ SSI Host

The ability Fer1L4, NL1 and NL2 loci to support recombinant gene expression and complete RMCE was then tested. Targeting vector pMF25 (FIG. 3A) and recombinase vector pMF4 (FIG. 3B) were co-transfected into 6 CHOK1SV GS-KO™ (FIG. 4: Fer1L4 loci: 7878 and 8086, NL1: 8096 and 9113, NL2: 9116 and 9115) single landing pad SSI hosts and incubated in glutamine-free medium to select for cells which have completed RMCE. These CHOK1SV GS-KO™ SSI pools were analyzed by flow cytometry prior to RMCE and after 11 days in glutamine-free medium (FIG. 4). As the landing pad in the clones (Landing Pad A, FIG. 2) contain the Hpt-eGFP reporter (detected in the green channel) and the pMF25 targeting vector contains DsRed reporter (detected in the yellow channel), successful RMCE was demonstrated by a change from +GFP, −YFP to −GFP, +YFP fluorescence.

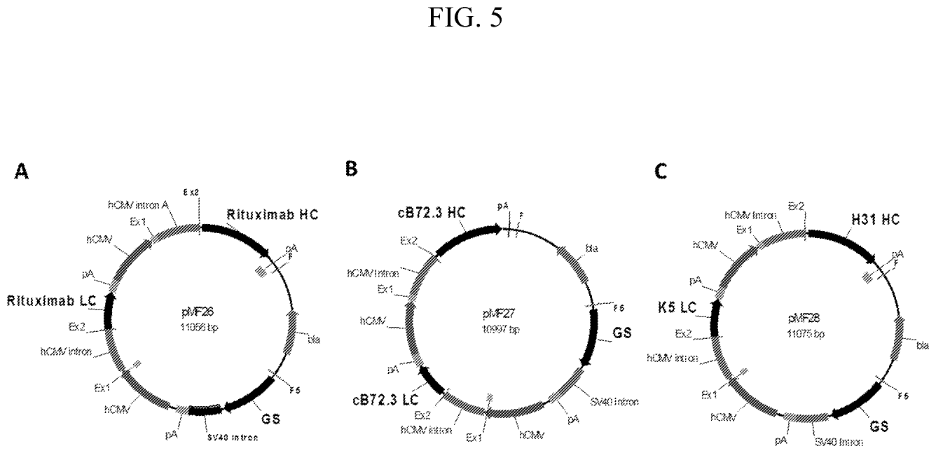

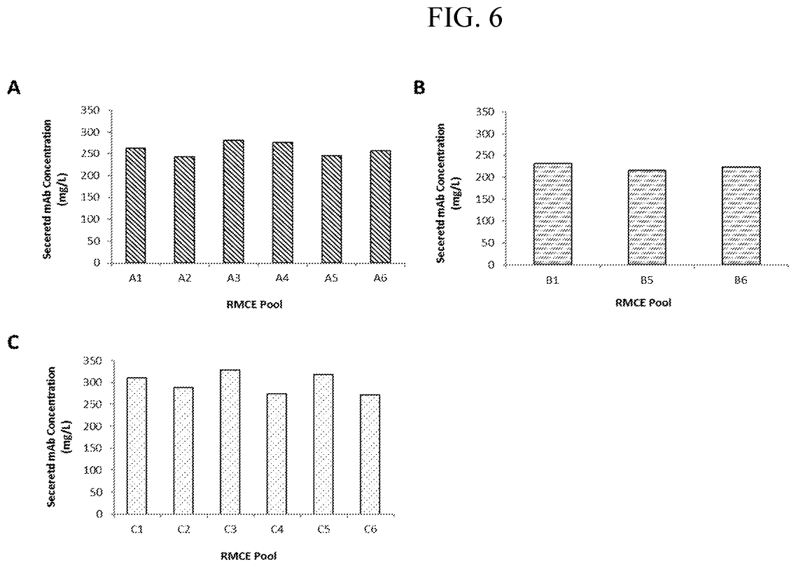

The SSI host clone 11434 (landing pad in the Fer1L4 loci, Landing Pad A: FIG. 2) was then tested for the ability to produce therapeutic mAbs. Vectors containing transcription units for rituximab, cB72.3 and H31K5 which target the Fer1L4 locus in clone 11434 were created (See FIG. 5). CHOK1SV GS-KO™ pools were then constructed and cultured in batch suspension culture for 8 days. The concentration of secreted mAb at harvest was determined by Protein A HPLC at harvest (See FIG. 6). These data show very consistent expression between replicated pools and between different mAbs (250-300 mg/L).

### Example 4: Evaluating Fer1L4 and NL1 in a Multisite CHOK1SV GS-KO™ SSI Host

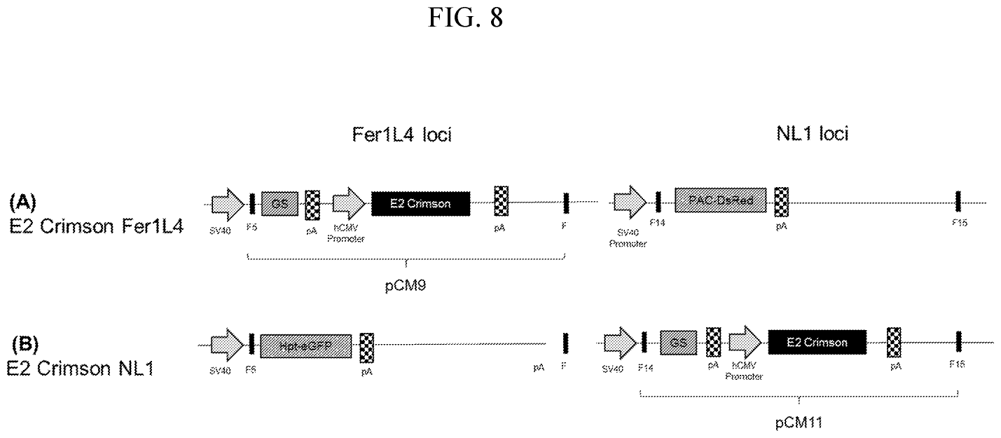

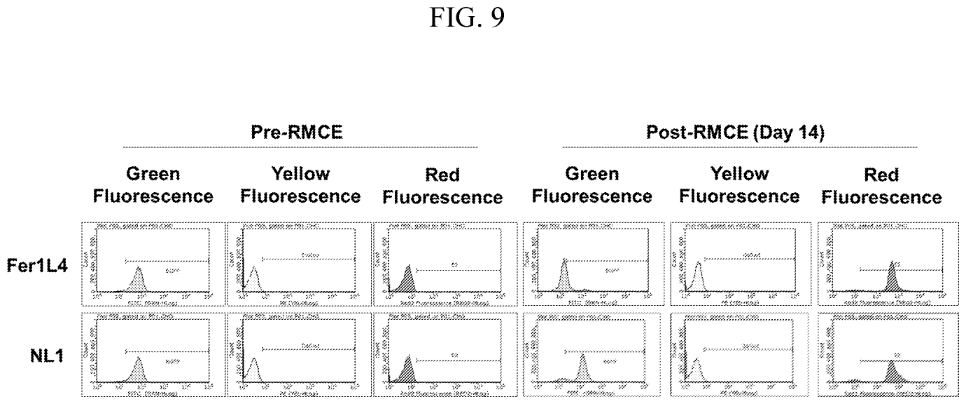

The 6 multisite hosts described in Example 2 were analyzed by flow cytometry to confirm the ability of loci to support recombinant gene expression (FIG. 7). The landing pad integrated at the Fer1L4 loci (FIG. 2: Landing Pad A) contains the Hpt-eGFP gene and the landing pad integrated at NL1 loci (FIG. 2: Landing Pad B) contains the PAC-DsRed gene. eGFP fluorescence was detected in the green channel and DsRed was detected in the yellow channel. The CHOK1SV GS-KO™ host (Host) was used as a negative control. Clone 11434 (landing pad in the Fer1L4 loci, Landing Pad A: FIG. 2) was used as a positive control for eGFP fluorescence. A CHOK1SV GS-KO™ pool expressing a DsRed gene which was generated by random integration (DsRed RI Control), was used as a positive control for Dsred fluorescence. As expected all 6 multisite CHOK1SV GS-KO™ SSI clones (12151, 12152, 12606, 12607, 12608 and 12609) had similar in eGFP and DsRed fluorescence intensities to that of 11434 and DsRed RI Control (FIG. 7). This demonstrated that both sites are capable of supporting expression of exogenous genes. Next the ability of the multi-site host to complete RMCE was confirmed. Targeting vectors pCM9 (FIG. 8A, contains E2 crimson expression cassette and targets landing pad A: Fer1L4) and pCM11 (FIG. 8B, contains E2 crimson expression cassette and targets landing pad A: NL1) were transfected into multi-site CHOK1SV GS-KO™ SSI host 12151. Pools were incubated in glutamine-free medium to select for cells which have completed RMCE. The no-FlpE controls did not recover with RMCE pools. These CHOK1SV GS-KO™ SSI pools were analyzed by flow cytometry prior to RMCE and after 14 days in glutamine-free medium (FIG. 8). As landing pad A in the CHOK1SV GS-KO™ SSI host (Landing Pad A, FIG. 2) contains the Hpt-eGFP reporter and the pCM9 targeting vector contains E2 crimson reporter (detected in the red channel) successful RMCE was demonstrated by a change from +GFP, −RFP to −GFP, +RFP fluorescence. Landing pad B in the CHOK1SV GS-KO™ SSI host (Landing Pad B, FIG. 2) contains the PAC-DsRed reporter however the DsRed signal was not detected on the flow cytometer (FIG. 9). pCM11 targeting vector contains E2 crimson reporter and therefore successful RMCE without the loss of Landing Pad A (containing Hpt-eGFP gene) was demonstrated by a change from +GFP, −RFP to +GFP, +RFP (FIG. 9).

To realize the benefit of the system for therapeutic proteins, the CHOK1SV™ SSI multisite hosts (See FIG. 2) were tested for the expression of therapeutic proteins outlined in Table 10. Experiments are separated into three phases; Phase 1: Tests the application of multisite SSI to increase qP; Phase 2: Tests the capability of multisite SSI to express a three-gene bispecific mAb across the two landing pads; Phase 3: To express ancillary genes in one site in order to aid the expression of a DtE protein encoded in the other site.

TABLE 10

| | | Therapeutic | |
|---|---|---|---|
| Phase | Rational | protein | Ancillary Gene |
| 1 | mAb genes in | rituximab, | N/A |
| | multiple SSIS | cB72.3 and | |
| | to increase qP | H31K5 etc. | |
| 2 | Bispecific mAb | Cergutuzumab | N/A |
| | and >2 chain | amunaleukin | |
| | DtE genes | etc. | |
| | expressed | | |
| | from SSIS | | |
| 3 | Ancillary gene | Infliximab | Lipid genes from |
| | expression to | etc. | Candidates identified in |
| | increase | | U.S. application No. |
| | product quality | | 62/322,621, incorporated |
| | of DtE | | herein by reference, the |
| | | | miRNA sponge vectors as |
| | | | found in Table 11, and |
| | | | any blood factor proteases. |

Phase 1:

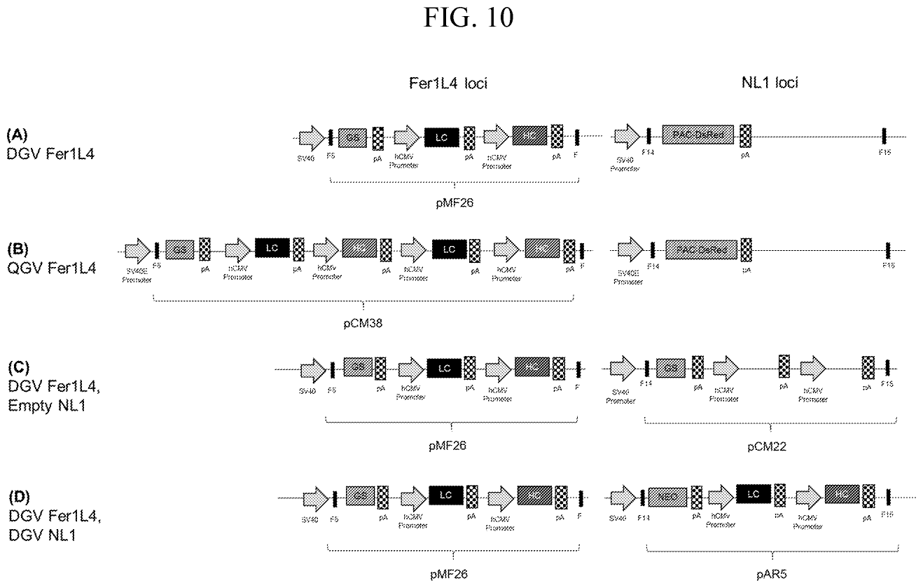

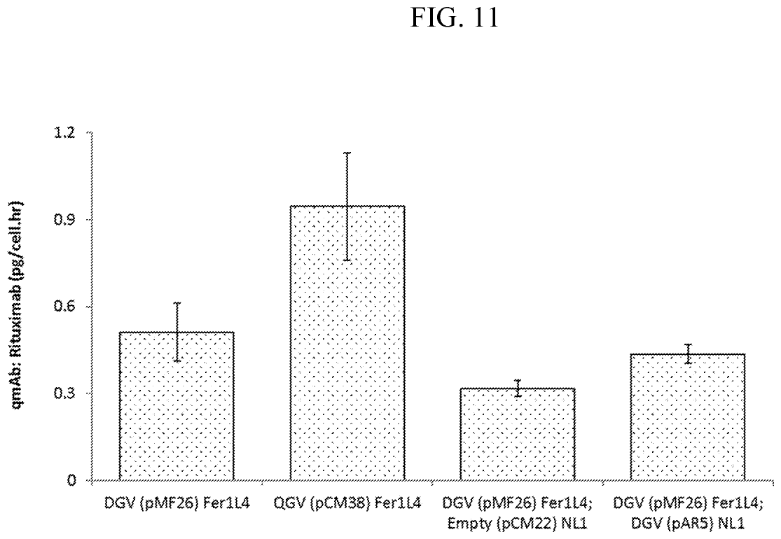

The limitation of some single site SSI systems is that a single copy of the necessary transcription units is not sufficient to generate suitable titers for clinical manufacturing. Therefore we have evaluated the option to use multisite SSI to increase the integrated copy number of mAb genes. Two approaches to increasing integrated copy number using the monoclonal antibody, rituximab were tested. In the first approach, a targeting vector was generated that contained four mAb expression cassettes (FIG. 10B: pCM38=2×HC and 2 LC) and targets landing pad A in the CHOK1SV GS-KO™ SSI host (Landing Pad A, FIG. 2A). This vector and the two mAb expression cassette equivalent vector (FIG. 10A: pMF26) were transfected separately (in duplicate) into the GS-KO SSI host clone 12151 (FIGS. 10A and B) and pools selected in glutamine-free medium (for a detailed transfection method see Example 2). In the second approach, a targeting vector that contained 2 mAb expression cassettes (FIGS. 10C and D: pAR5=1×HC and 1×LC) in addition to the neomycin phosphotransferase gene (NEO) which targeted landing pad B (Landing Pad B, FIG. 2A) were generated. A version of pAR5 which lacked mAb genes was also created and referred to as pCM22 (FIG. 10C). CHOK1SV GS-KO™ SSI pools transfected with pMF26 and selected in glutamine-free medium were then transfected (in duplicate) with either pAR5 (FIG. 10D) or pCM22 (FIG. 10C) (for a detailed transfection method see Example 2), incubated in the presence of pMF4 (FIG. 3B) for 24 hours and then selected in 400 μg/mL Geneticin (G418). Consistent with selection in glutamine free medium, No-FlpE controls cultured in the presence of G418 did not recover with RMCE pools. Following recovery from selection, the eight RMCE pools were sub-cultured and then progressed to an 8-day batch culture. The viable cell concentration was determined at days 4, 6 and 8 using a Vicell cell counter and concentration of secreted mAb at harvest was determined by ForteBio Octet using Protein A sensors. Cell specific production rate of Rituximab (qmAb at harvest) was calculated (See FIG. 11). These data demonstrated that both scenarios are viable options for increasing cell specific mAb production rates from the CHOK1SV GS-KO™ SSI host.

Phase 2:

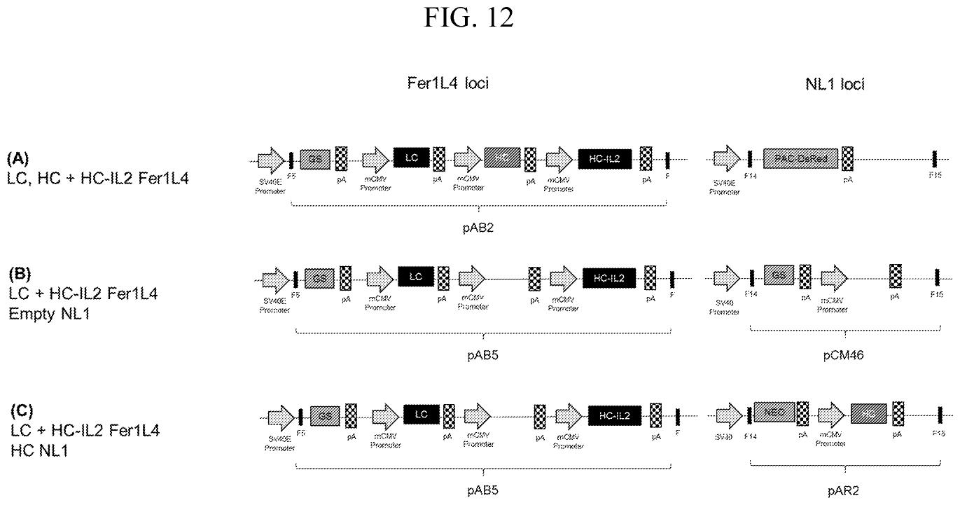

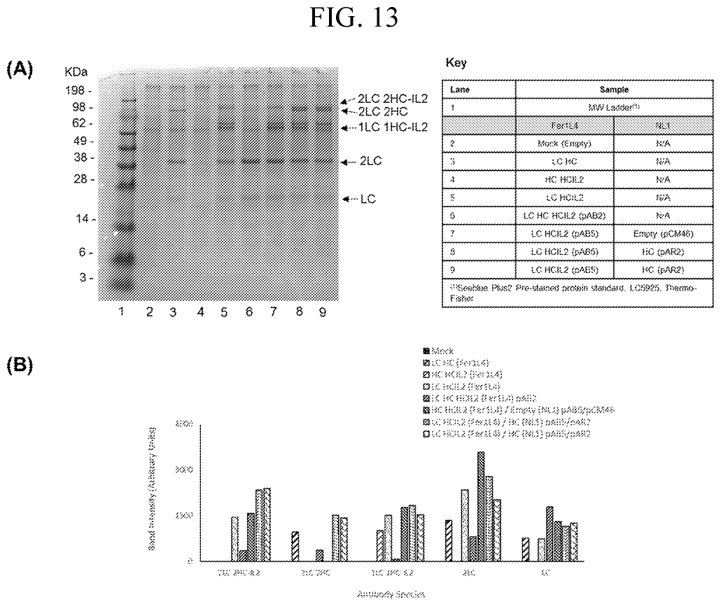

For the majority of next generation antibodies (e.g. tetravalent bispecific antibodies) the assembly of multiple heavy or light chains is a recurrent problem. In order to obtain optimal product quality with very few unwanted side products, the selection of an appropriate CHO clone expressing as many as four antibody chains in a stable and reproducible is beneficial. As a result, a large amount of product analytics utilizing ELISAs, RP-HPLC or CD-SDS during clone selection is often required. However, in a multisite SSI cell line, the genes encoding a multi chain protein are driven with individual promoters and are spatially separated across at least two sites. This ensures that copy number and relative expression of individual chains are consistent as early as transfectant pools and enabling empirical fine-tuning of the availability of individual polypeptide chains through promoter strength or copy number manipulation of the encoding transcription units. The advantage of this are that product quality is more consistent, reducing the proportion of misfolded multi-chain recombinant protein produced from SSI-generated pools and cell lines, dramatically recuing the need for early stage assessments. Therefore we have evaluated the opportunity to use multisite SSI to express the cergutuzumab amunaleukin (CEA-IL2v) which is encoded by three genes: LC, HC and HC-IL2 (Klein et al., Oncoimmunology 6: 3 (2017). We tested two approaches for the insertion of these three genes into the genome of the CHOK1SV GS-KO™ SSI host. In this experiment (Phase 2) the mouse cytomegalovirus first immediate early gene (IE1) (mCMV) promoter (EP 1525320) in combination with human intron A (Addison et al., Journal of General Virology, 78 (1997) was used, instead of the hCMV promoter, to drive expression of recombinant protein. In the first approach we inserted cergutuzumab amunaleukin LC, HC and HC-IL2 into landing pad A using targeting vector pAB2 (FIG. 12A). Selection was in the absence of glutamine as described in Example 2. In the second approach CHOK1SV GS-KO™ SSI host was transfected with pAB5 (FIG. 12B) which contains expression units for cergutuzumab amunaleukin LC and HC-IL2 targeting landing pad A (FIG. 2). Selection was in the absence of glutamine as described in Example 2. Subsequently we generated a targeting vector that contained a single cergutuzumab amunaleukin HC expression cassette (FIG. 12C: pAR2) in addition to the neomycin phosphotransferase selection marker gene (NEO) which targeted landing pad B (Landing Pad B, FIG. 2A). A version of pAR2 which lacked mAb gene was also created and referred to as pCM46 (FIG. 12B: pCM46). CHOK1SV GS-KO™ SSI pools transfected with pAB5 and selected in glutamine-free medium were then transfected (in duplicate) with either pCM46 or pAR2 (for a detailed transfection method see Example 2) and selected in medium containing 400 μg/mL Geneticin (G418). Control pools were also constructed: a ‘Mock’ transfection with an empty version of pAR2, and three pools, each which lacked one of the three genes encoding cergutuzumab amunaleukin (LC+HC, LC+HC-IL2 and HC+HC-IL2), to be used for identifying CEA-IL2v antibody species. Following recovery from selection, the RMCE pools were sub-cultured and then progressed to an 8-day batch culture. The viable cell concentration was determined at days 4, 6 and 8 using a Vicell cell counter. Cergutuzumab amunaleukin assembly species were determined by non-reduced analysis of supernatants on 10% Bis-Tris SDS PAGE gels (FIG. 13A) with subsequent densitometry analysis (ImageJ software) (FIG. 13B). These data showed that when cergutuzumab amunaleukin LC, HC and HC-IL2 genes are inserted into landing pad A the expression of individual chains in low and only small amounts of what is believed to be fully assembled product can be seen. In contrast when cergutuzumab amunaleukin LC and HC-IL2 are targeted to landing pad A and HC is targeted to landing pad B, the expression of individual chains is increased and more fully assembled product is detected. The reproducibility of this result (in replicate pools) supports the opportunity of using a multi-site SSI approach to tune relative quantities of each chain to increase product quality. The genes required for bi-specific assembly have been separated across two sites (and not topped up in the second site) and demonstrated an improvement in expression.

Phase 3:

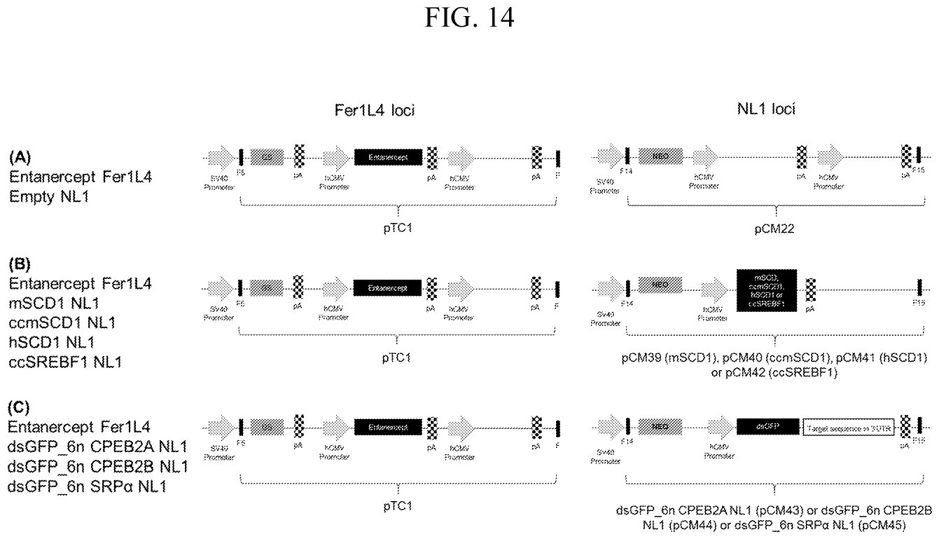

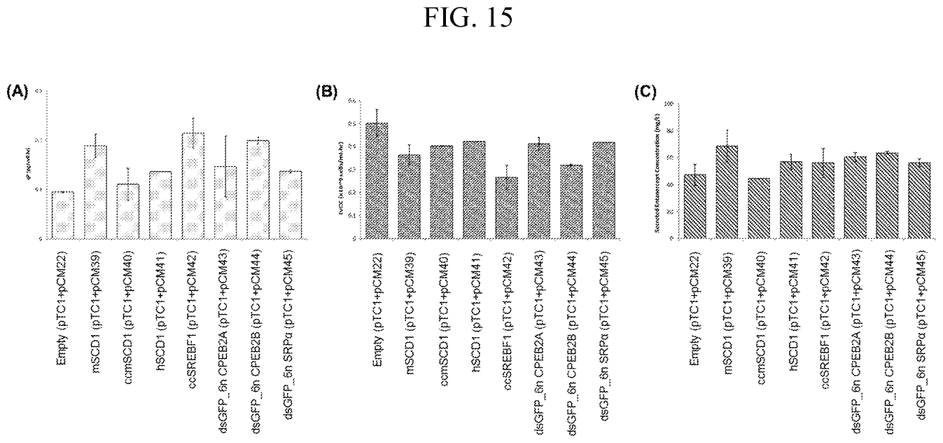

Expression of endogenous proteins which aims to increase of the secretion capacity of the CHOK1SV GS-KO™ cell line is a proven approach to increase product titers. Candidates identified from PCT application WO2015018703 A1 and those in Tables 10, 11 and 12 were evaluated in the multisite SSI cell line. In order to test this concept, a vector expressing etanercept (FIGS. 14A, B and C: pTC1) which targeted landing pad A was constructed. This was transfected separately (in duplicate) into the CHOK1SV GS-KO™ SSI host (FIG. 14) and pools selected in glutamine-free medium (for a detailed transfection method see Example 2). pCM39 to pCM45 contain expression units for ancillary genes under the control of the human cytomegalovirus major immediate-early (hCMV) promoter (with its first Intron A) and targets landing pad B (FIG. 14) (Table 11 and 12). In pCM39-42, variants of stearoyl-CoA desaturase-1 (Scd1) and Sterol regulatory element-binding protein 1 (SREBF1) were targeted to the NL1 locus. In pCM43, 44 and 45 a short-lived GFP (or dsGFP) designed with a c-terminus PEST sequence (SEQ ID NO: 38) from sequence gb:CQ871827 was constructed and 6 copies of the miR Target Sequence inserted into 3′UTR for CPEB2A (pCM43), CEPB2B (pCM44) and SRPα (pCM45). These targeting vectors contain the neomycin phosphotransferase gene (NEO) and 24 hours following transfection (with pMF4) of each vector into duplicate pTC01 transfected pools, selection was achieved using medium supplemented with 400 μg/mL G418. Following successful recovery of pools and at least one subculture, a 8-batch culture was established. The viable cell concentration was determined at days 4, 6 and 8 using a Vicell cell counter and concentration of secreted mAb at harvest was determined by ForteBio Octet using Protein A sensors. Integral of viable cell concentration (IVCC), cell specific production rate (qP at harvest) and secreted etanercept concentration are presented (See FIG. 15). These data demonstrated elevated secreted etanercept amounts with expression of mouse SCD1 (mSCD1) and sponge vectors bearing CEPB2A (dsGFP_6n CPEB2A), CPEB2B (dsGFP_6n CPEB2B) and SRPα (dsGFP_6n SRPα) miR binding site in 3′UTR.

TABLE 11

| | Vector | | |
|---|---|---|---|
| Ancillary Gene | Annotation | Species | Reference |
| stearoyl-CoA | mSCD1 | Mus musculus | As described in |
| desaturase 1 | | | U.S. application |
| (SCD1) | | | No. 62/322,621 |
| | ccmSCD1 | Mus musculus | As described in |
| | | codon optimized for | U.S. application |
| | | Cricetulus griseus | No. 62/322,621 |
| | hSCD1 | Homo sapiens | NM_005063.4 |
| sterol regulatory | SREBF1 | Cricetulus griseus | As described in |
| element-binding | | | U.S. application |
| protein 1 | | | No. 62/322,621 |

TABLE 12

| | Target Gene | Sponge Sequence | |
|---|---|---|---|
| | (miR binding | (miR Target Sequence in | Copies of 3′UTR target |
| miR | site in 3′UTR) | Gene 3′UTR | site |
| miR-15b and miR- | CPEB2A | 5′-aggggcaacacagtctgctgcta-3′ | 1, 3, 6, 9 and 12 |
| 16-1 | | (SEQ ID NO: 39) | |
| miR-15b and miR- | CPEB2B | 5′-aagctgtattagctttgctgcta-3′ | |
| 16-1 | | (SEQ ID NO: 40) | |
| miR-34c | SRPRα | 5′-taatcatgttacaatcactgcc-3′ | |
| | | (SEQ ID NO: 41) | |
| miR-708 | CNTFR | 5′-caccatcagattataagctcctg-3′ | |
| | | (SEQ ID NO: 42) | |
| miR-186 | EIF3A | 5′-cagtctaaattgaattcttta-3′ | |
| | | (SEQ ID NO: 43) | |

### Example 5: Transfer of Loci Between CHOK1SV and HEK293 Cells Lines

In order to generate a HEK293 SSI host, CHOK1SV derived vector integration sites and landing pad locations were BLAT searched against the human genome (version: March 2006 (NCBI36/hg18)) using a stand-alone copy of the University of California Santa Cruz (UCSC) human genome data base. This identifies sequences of 95% (and greater) similarity, in at least 25 base pairs of CHOK1SV sequence. Regions of similarity were visualized in IGV viewer (Broad institute version 2.4). Crispr-Cas9 gRNA were design using an in house Crispr-Cas9 design tool. CHOK1SV and HEK293 loci are summarized in Table 1.

What is claimed is:

1. A mammalian cell comprising at least two distinct recombination target sites (RTS) wherein two RTS are chromosomally-integrated within the NL1 locus or the NL2 locus.

2. The cell of claim 1, comprising two distinct RTS.

3. The cell of claim 1, wherein the two distinct RTS are chromosomally-integrated within the NL1 locus.

4. The cell of claim 1, wherein the two distinct RTS are chromosomally-integrated within the NL2 locus.

5. The cell of claim 1, comprising four distinct RTS.

6. The cell of claim 1, where the four distinct RTS are chromosomally-integrated on the same locus.

7. The cell of claim 6, wherein two RTS are chromosomally-integrated on a separate locus.

8. The cell of claim 7, wherein the separate locus is the Fer1L4 locus.

9. The cell of claim 5, wherein two distinct RTS are chromosomally-integrated within the NL1 locus and two distinct RTS are chromosomally-integrated within the NL2 locus.

10. The cell of claim 1, comprising six distinct RTS.

11. The cell of claim 10, where at least four distinct RTS are chromosomally-integrated on the same locus.

12. The cell of claim 10, wherein at least two distinct RTS are chromosomally-integrated on a separate locus.

13. The cell of claim 12, wherein the separate locus is the Fer1L4 locus.

14. The cell of claim 10, wherein at least two distinct RTS are chromosomally-integrated within the NL1 locus and at least two distinct RTS are chromosomally-integrated within the NL2 locus.

15. The cell of any one of claims 1 to 14, wherein at least one of the RTS is a frt site, a lox site, a rox site, or an att site.

16. The cell of any one of claims 1 to 15, wherein at least one of the RTS is selected from among SEQ ID NOs.: 1-30.

17. The cell of any one of claims 1 to 16, wherein the mammalian cell is a mouse cell, a human cell, a Chinese hamster ovary (CHO) cell, a CHO-K1 cell, a CHO-DXB11 cell, a CHO-DG44 cell, a CHOK1SV™ cell including all variants, a CHOK1SV GS-KO™ (glutamine synthetase knockout) cell including all variants, a HEK cell, a HEK293 cell including adherent and suspension-adapted variants, a HeLa cell, or a HT1080 cell.

18. The cell of claim 17, wherein the cell is a HEK cell or a HEK293 cell including adherent and suspension-adapted variants.

19. The cell of any one of claims 1 to 18, further comprising a first gene of interest, wherein the first gene of interest is chromosomally-integrated.

20. The cell of claim 19, wherein the first gene of interest comprises a reporter gene, a selection gene, a gene of therapeutic interest, an ancillary gene, or a combination thereof.

21. The cell of claim 20, wherein the gene of therapeutic interest comprises a gene encoding a difficult to express protein.

22. The cell of claim 21, wherein the difficult to express protein is selected from the group consisting of a Fc-fusion protein, an enzyme, a membrane receptor, or a monoclonal antibody.

23. The cell of claim 22, wherein the monoclonal antibody is a bi-specific monoclonal antibody or a tri-specific monoclonal antibody.

24. The cell of any one of claims 18 to 23, wherein the first gene of interest is located between two of the RTS.

25. The cell of any one of claims 18 to 24, wherein the first gene of interest is located within the NL1 locus.

26. The cell of any one of claims 1 to 25, further comprising a second gene of interest, wherein the second gene of interest is chromosomally-integrated.

27. The cell of claim 26, wherein the second gene of interest comprises a reporter gene, a selection gene, a gene of therapeutic interest, an ancillary gene, or a combination thereof.

28. The cell of claim 27, wherein the gene of therapeutic interest comprises a gene encoding a difficult to express protein.

29. The cell of claim 28, wherein the difficult to express protein is selected from the group consisting of a Fc-fusion protein, an enzyme, a membrane receptor, or a monoclonal antibody.

30. The cell of any one of claims 26 to 29, wherein second gene of interest is located between two of the RTS.

31. The cell of any one of claims 26 to 30, wherein the second gene of interest is located within the NL1 locus or the NL2 locus.

32. The cell of any one of claims 26 to 31, wherein the first gene of interest is located within the NL1 locus, and the second gene of interest is located within the NL2 locus.

33. The cell of any one of claims 1 to 32, further comprising a third gene of interest, wherein the third gene of interest is chromosomally-integrated.

34. The cell of claim 33, wherein the third gene of interest comprises a reporter gene, a selection gene, a gene of therapeutic interest, an ancillary gene, or a combination thereof.

35. The cell of claim 34, wherein the gene of therapeutic interest comprises a gene encoding a difficult to express protein.

36. The cell of any one of claims 33 to 35, wherein the third gene of interest is located between two of the RTS.

37. The cell of any one of claims 33 to 36, wherein the third gene of interest is located within the NL1 locus or the NL2 locus.

38. The cell of any one of claims 33 to 37, wherein the third gene of interest is located within a locus distinct from the NL1 locus and the NL2 locus.

39. The cell of any one of claims 33 to 38, wherein the first gene of interest, the second gene of interest, and the third gene of interest are within three separate loci.

40. The cell of any one of claims 33 to 39, wherein
a. at least one of the first gene of interest, the second gene of interest, and the third gene of interest is within the NL1 locus and
b. at least one of the first gene of interest, the second gene of interest, and the third gene of interest is within the NL2 locus.

41. The cell of any one of claims 1 to 40, further comprising a site-specific recombinase gene.

42. The cell of claim 41, wherein the site-specific recombinase gene is chromosomally-integrated.

43. A mammalian cell comprising at least four distinct, wherein the cell comprises:
a. at least two distinct RTS are chromosomally-integrated within the NL1 locus or NL2 locus;
b. a first gene of interest is integrated between the at least two RTS of (a), wherein the first gene of interest comprises a reporter gene, a gene encoding a difficult to express protein, an ancillary gene or a combination thereof; and
c. a second gene of interest is integrated within a second chromosomal locus distinct from the locus of (a), wherein the second gene of interest comprises a reporter gene, a gene encoding a difficult to express protein, an ancillary gene or a combination thereof.

44. A mammalian cell comprising at least four distinct recombination target sites (RTS), wherein the cell comprises:
a. at least two distinct RTS are chromosomally-integrated within the Fer1L4 locus;
b. at least two distinct RTS are chromosomally-integrated within the NL1 locus or the NL2 locus;
c. a first gene of interest is chromosomally-integrated within the Fer1L4 locus, wherein the first gene of interest comprises a reporter gene, a gene encoding a difficult to express protein, an ancillary gene or a combination thereof; and
d. a second gene of interest is chromosomally-integrated within the within the NL1 locus or NL2 locus of (b), wherein the second gene of interest comprises a reporter gene, a gene encoding a difficult to express protein, an ancillary gene or a combination thereof.

45. A mammalian cell comprising at least six distinct recombination target sites (RTS), wherein the cell comprises:
a. at least two distinct RTS and a first gene of interest are chromosomally-integrated within the Fer1L4 locus;
b. at least two distinct RTS and a second gene of interest are chromosomally-integrated within the NL1 locus; and
c. at least two RTS and a third gene of interest are chromosomally-integrated within the NL2 locus.

46. The cell of any one of claims 43-45, wherein the cell is a HEK cell or a HEK293 cell including adherent and suspension-adapted variants.

47. A method for producing a recombinant protein producer cell comprising:
a. providing a cell that comprises at least four distinct recombination target sites (RTS) and a gene encoding a site-specific recombinase, wherein at least two distinct RTS are chromosomally-integrated within the NL1 locus and at least two distinct RTS are chromosomally-integrated within the NL2 locus;
b. transfecting the cell of (a) with a first vector comprising an exchangeable cassette encoding a first gene of interest and a second vector comprising an exchangeable cassette encoding a second gene of interest;
c. integrating the first exchangeable cassette within the NL1 locus and the second exchangeable cassette within the NL2 locus; and
d. selecting a recombinant protein producer cell comprising the first exchangeable cassette and the second exchangeable cassette integrated into the chromosome.

48. The method of claim 47, wherein the first gene of interest comprises a reporter gene, a selection gene, a gene of therapeutic interest, an ancillary gene, or a combination thereof.

49. The method of claim 48, wherein the gene of therapeutic interest comprises a gene encoding a difficult to express protein.

50. The method of claim 49, wherein the difficult to express protein consists of a Fc-fusion protein, an enzyme, a membrane receptor, or a monoclonal antibody.

51. The cell of claim 50, wherein the monoclonal antibody is a bi-specific monoclonal antibody or a tri-specific monoclonal antibody.

52. The method of any one of claims 45 to 51, wherein the first gene of interest is located between two of the RTS.

53. The method of any one of claims 47 to 52, wherein the second gene of interest comprises a reporter gene, a selection gene, a gene of therapeutic interest, an ancillary gene, or a combination thereof.

54. The method of claim 53, wherein the gene of therapeutic interest comprises a gene encoding a difficult to express protein.

55. The method of any one of claims 47 to 54, wherein the second gene of interest is located between two of the RTS.

56. A method for producing a recombinant protein producer cell comprising:
a. providing a cell that comprises at least at least four distinct recombination target sites (RTS) and a gene encoding a site-specific recombinase, wherein at least two distinct RTS are chromosomally-integrated within the Fer1L4 locus, and at least two distinct RTS are chromosomally-integrated within the NL1 locus or the NL2 locus;
b. transfecting the cell of (a) with a first vector comprising an exchangeable cassette encoding a first gene of interest and a second vector comprising an exchangeable cassette encoding a second gene of interest;
c. integrating the first exchangeable cassette within the Fer1L4 locus and the second exchangeable cassette within the NL1 locus or the NL2 locus; and
d. selecting a recombinant protein producer cell comprising the first exchangeable cassette and the second exchangeable cassette integrated into the chromosome.

57. The method of claim 56, wherein the cell is a HEK cell or a HEK293 cell including adherent and suspension-adapted variants.

58. The method of claim 56, wherein the first gene of interest comprises a reporter gene, a selection gene, a gene of therapeutic interest, an ancillary gene, or a combination thereof.

59. The method of claim 58, wherein the gene of therapeutic interest comprises a gene encoding a difficult to express protein.

60. The method of claim 59, wherein the difficult to express protein consists of a Fc-fusion protein, an enzyme, a membrane receptor, or a monoclonal antibody.

61. The cell of claim 60, wherein the monoclonal antibody is a bi-specific monoclonal antibody or a tri-specific monoclonal antibody.

62. The method of any one of claims 56 to 61, wherein the first gene of interest is located between two of the RTS.

63. The method of any one of claims 56 to 62, wherein the second gene of interest comprises a reporter gene, a selection gene, a gene of therapeutic interest, an ancillary gene, or a combination thereof.

64. The method of claim 63, wherein the gene of therapeutic interest comprises a gene encoding a difficult to express protein.

65. The method of any one of claims 56 to 66, wherein the second gene of interest is located between two of the RTS.

66. A method for producing a recombinant protein producer cell comprising:
a. providing a cell that comprises at least at least six distinct recombination target sites (RTS) and a gene encoding a site-specific recombinase, wherein at least two distinct RTS are chromosomally-integrated within the Fer1L4 locus, and at least two distinct RTS are chromosomally-integrated within the NL1 locus, and at least two distinct RTS are chromosomally-integrated within the NL2 locus;
b. transfecting the cell of (a) with a first vector comprising an exchangeable cassette encoding a first gene of interest, a second vector comprising an exchangeable cassette encoding a second gene of interest, and a third vector comprising an exchangeable cassette encoding a third gene of interest;
c. integrating the first exchangeable cassette within the Fer1L4 locus, the second exchangeable cassette within the NL1 locus, and the third exchangeable cassette within the NL2 locus; and
d. selecting a recombinant protein producer cell comprising the first exchangeable cassette, the second exchangeable cassette and the third exchangeable cassette integrated into the chromosome.
